(19) United States
(12) Patent Application Publication
(10) Pub. No.: US 2023/0383255 A1
(43) Pub. Date: Nov. 30, 2023

(54) GENETICALLY ENGINEERED DENDRITIC CELLS TO ACTIVATE PROTEIN SPECIFIC T CELLS FOR THE TREATMENT OF VIRAL AND OTHER PATHOGENIC INFECTIONS

(71) Applicant: NeyroblastGX LLC,, Murrieta, CA (US)

(72) Inventors: Aynun N. Begum, Murrieta, CA (US); Alexander V. Michkov, Murrieta, CA (US)

(21) Appl. No.: 18/324,105

(22) Filed: May 25, 2023

Related U.S. Application Data
(60) Provisional application No. 63/365,327, filed on May 25, 2022.
(60) Provisional application No. 63/366,127, filed on Jun. 9, 2022.

(51) Int. Cl. C12N 5/0784; C07K 14/165; A61P 31/14

(52) U.S. Cl. CPC C12N 5/0639; C07K 14/165; A61P 31/14; C12N 2510/00; C12N 2506/45; C12N 2513/00; C12N 2501/165; C12N 2501/155; C12N 2501/22; C12N 2501/2304

(57) ABSTRACT

Provided are genetically engineered DC probes/epitopes that are able to stimulate high numbers of a pathogenic or viral, or degenerative protein (such as the functional spike (Sp), membrane (M), and nucleocapsid (N) protein and amyloid beta and tau protein) and produce protein-specific CD4⁺ and CD8⁺ T cells ex vivo, which can then be adaptively administered to patients to treat a variety of pathogenic infections, degenerative disorder, including viral infections.

Appl. No. 63/365,327 or 63/366,127

Provisional Application Under 35 USC 111(b): May 25, 2022 and Jun. 9, 2022

### FIELD OF THE INVENTION

The present invention relates to dendritic cells (DCs) from human induced pluripotent stem cells (HiPSCs) and DCs that are genetically modified to activate protein-specific T cells, such as to activate SARS-CoV-2 protein-specific T cells to treat COVID-19 or to treat other newly emerging viruses, pathogens, other infectious organisms and degenerative disease induced by pathological proteins.

### BACKGROUND

Various patents, patent applications, and publications are cited herein, and these disclosures are incorporated by reference in their entirety. However, the citation of any reference herein should not be construed as an admission that such reference is available as “Prior Art” to the present application.

SARS-CoV-2 virus-induced infections continue to affect people in the United States (US) and worldwide. According to the Centers for Disease Control (CDC), coronavirus deaths in the US rose to over 1.1 million.¹ Theoretically, the COVID-19 pandemic is over because of vaccine and booster development. Nevertheless, the COVID-19 pandemic is not over and will likely become endemic soon. The CDC reported that as of Jan. 4, 2023, the current 7-day average of weekly new cases (67,243) increased by 16.2% compared with December 2022², proving the COVID-19 pandemic continues. Now COVID-19 exceeded 101 million in the US², which warrants a broad spectrum of therapies or treatments against SARS-CoV-2 variants. In this context, we develop a genetically engineered therapeutic DC probe from human induced pluripotent stem cells (HiPSCs)-derived from dendritic cells (DC)s, which can activate highly proliferative and protective CD4⁺ and CD8⁺ T cells against the current and new variants of SARS-CoV-2. DC-COV19 is a genetically engineered DC generated by integrating SARS-CoV-2-specific viral constructs into the DC genome to activate T cells with higher sensitivity and specificity. The DC-engineered viral constructs of the invention, including human leukocyte antigen—DR or DRA isotype (HLA-DR or HLA-DRA) covalently linked to the spike protein, or portions of the spike protein, including sub-units thereof, causes DCs to generate robust CD4⁺ and CD8⁺ T cells in patients regardless of race and ethnicity since HLA-DRA is found in 98% of the human population.3,4 Thus, the HLA-DRA combined spike protein of SARS-CoV-2 constructs of the invention produces a higher frequency of protective CD4+ T cells in clinical samples. Evidence suggests that COVID-19 patients severely exhaust their CD4+ and CD8+ T cells, especially in moderate to severe cases.5,6,7,8,9 In addition, the current mRNA-based COVID-19 vaccine does not enhance the SARS-CoV-2 specific T cells if patients lack these in their systems. Even the mRNA vaccine has various adverse side effects, as reported recently.10,11 Thus, the present inventors developed an alternative safe, cost-effective, and easy-to-use therapy for individual use that produces even personalized levels of treatment.

Dendritic cells are central to the initiation of primary immune responses. They are the only antigen-presenting cell capable of stimulating naive T cells, and hence they are pivotal in the generation of adaptive immunity.¹²

Dendritic cells (DC) are a type of antigen-presenting cell (APC) that play an essential role in the adaptive immune system. The primary function of DCs is to present antigens; therefore, the cells are sometimes referred to as “professional” APCs. Dendritic cells are so named because they develop branched projections called “dendrites” during development to maximize their surface area and increase exposure to antigens. Dendritic cells are found in tissue that has contact with the outside environment, such as lung mucosa, skin epithelial cells, and the nose and gastrointestinal tract linings. These regions form the interface between the body and the environment and are constantly exposed to foreign proteins and pathogens. Immature forms of DCs are also found in the blood. Once activated DCs move to the lymph tissue to interact with T and B cells and help shape the adaptive immune response. In the intraepithelial tissue of the respiratory system, they can be found in very high densities (500-1,000 cells per mm²). Their densities depend on the level of antigen exposure, being highest in the proximal airways and decreasing towards the distal airways and alveoli.¹³

Some prior publications have suggested the possibility of dendritic cell (DC)-based vaccines for SARS-CoV-2, but to date, the present inventors are not aware of any successful such vaccines.

### SUMMARY OF THE INVENTION

The present invention is directed to a genetically engineered DC probe/epitope that is able to stimulate high numbers of a pathogenic or viral protein (such as the functional spike (Sp), membrane (M), and nucleocapsid (N) protein) and produce protein-specific CD4⁺ and CD8⁺ T cells ex vivo, which can then be adaptively administered to patients to treat a variety of pathogenic infections, including viral infections. Our exogenously induced CD4⁺ and CD8⁺ T cells are autogenic, safe, robust, and cost-effective T cell-based immunotherapeutic strategies to stop or reduce not only current SARS-CoV-2 infection but also new SARS-CoV-2 and other pathogen variants or degenerative diseases induced by pathogenic proteins. Besides, DCs engineered like the DC-COV19 of the invention can also be used for the treatment of various pathogens, viruses, bacteria, and parasites. For example, the present invention can produce genetically engineered DCs for the treatment of cytomegalovirus (CMV) or the polyomavirus family beta polyomavirus (BK) virus for potential delivery back into the patients to reduce or stop the risk of transplant-related infections, chronic infection, and Long COVID (FIG. 1). Thus, the highly innovative genetically engineered DC probe of the invention can be used to treat a broad spectrum of viral and other pathogens where traditional treatment or vaccines pose significant risks, such as chronic infections, immunosuppressive disorders, and older populations.

In one embodiment, the present invention is directed to a genetically engineered dendritic cell comprising a vector comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

In another embodiment, the pathogenic protein is a viral surface or membrane protein.

In a further embodiment, the protein is a viral protein from SARS-CoV 2, cytomegalovirus (CMV), BK virus (BKV).

In another embodiment, the protein is the SARS-CoV 2 spike protein or a fragment thereof.

In a yet further embodiment, the protein is at least one of the S1 and S2 subunits of SARS-CoV-2, or a fragment thereof.

In another embodiment, the invention is directed to a kit comprising genetically engineered dendritic cells comprising a vector comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

In a further embodiment, the dendritic cells in the kit are human dendritic cells derived from HiPSCs.

In another embodiment, the invention is directed to a polynucleotide construct comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

In a further embodiment, the pathogenic protein encoded by the construct is a viral surface or membrane protein.

In another embodiment, the protein is a viral protein from SARS-CoV 2, cytomegalovirus (CMV), BK virus (BKV), or the vial protein is the spike peptide (Spep) from SARS-CoV 2, or a fragment thereof, including at least one of the S1 and S2 subunit of SARS-CoV2, or a fragment thereof.

In another embodiment, the present invention is directed to a method for producing genetically engineered dendritic cells, the method comprising:
(a) culturing human induced pluripotent stem cells (HiPSCs) in a first culture medium comprising mesodermal growth factors, consisting of human recombinant (rh) bone morphogenetic protein 4 (BMP4), rh vascular endothelial growth factor (VEGF), rh stem cell factor (SCF), and rh granulocyte-macrophage colony-stimulating factor (GM-CSF) to produce 3-dimensional spheroid cells;
(b) adding further factors to said first culture medium, wherein said further factors comprise rh BMP4, rh VEGF, rh SCF, and rh GM-CSF;
(c) separate the 3-dimensional spheroid cells from said further factors, and culture said 3-dimensional spheroid cells in a second culture medium that comprises IL-4 to produce immature dendritic cells (iDCs);
(d) separate said iDCs from said second culture medium and culture said iDCs in a third culture medium comprising rhGM-CSF, rhIL-4, rhTNF-α, rhIFN-γ, prostaglandin E2 (PGE2), and rhIL-1β to produce 3-dimensional spheroids of fully functional mature dendritic cells (mDCs); and
(e) transfecting said mDCs dendritic cell with a vector comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

In a further embodiment of the method of the invention, the pathogenic protein is a viral surface or membrane protein, including a viral protein from SARS-CoV 2, cytomegalovirus (CMV), BK virus (BKV).

In a further embodiment of the method, the protein is the spike peptide (Spep) from SARS-CoV 2, or a fragment thereof, or at least one of the S1 and S2 subunits of SARS-CoV 2, or a fragment thereof.

In another embodiment, the present invention is directed to a method for producing protein-specific T cells, which comprises co-culturing T cells with genetically engineered dendritic cells comprising a vector comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

In a further embodiment of the method for the production of protein-specific T cells, the co-cultured T-cells are obtained from a patient or are from a source other than a patient to be treated with the protein-specific T cells.

In another embodiment, the present invention is directed to a method for treating a patient having a pathogen infection which comprises administering to a patient an effective amount of protein-specific T cells produced by co-culturing T cells with genetically engineered dendritic cells comprising a vector comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

In a further embodiment, the protein-specific T cells are administered intravenously to the patient.

In a further embodiment, the present invention relates to a T cell treatment preparation comprising protein-specific T cells produced by co-culturing T cells with genetically engineered dendritic cells comprising a vector comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof; and a pharmaceutically acceptable carrier, diluent or medium.

### BRIEF DESCRIPTION OF DRAWINGS

FIG. 1 graphically depicts the stepwise generation of genetically engineered DC probes from HiPSCs.

FIG. 2A provides images of naive DCs generated from HiPSCs.

FIG. 2(B) shows the results of tests on the DC's phagocytic activity with FITC-IgG.

FIG. 3(A) provides images to show DC characterization by ICC.

FIG. 3(B) provides graphic results from flow cytometry to show the DC's functional activities and morphological characteristics.

FIG. 4(A) provides a graphic depiction of SARS-CoV-2 spike protein (Sp) structure that enters human cells.

FIG. 4(B) describes the eight inserts used to prepare the genetically engineered DCs, including the full-length Sp, the subunits S1 and S2, and the HLA-DRA segment, wherein two constructs were labeled with GFP.

FIG. 4(C) shows the results of expression for the constructs.

FIG. 4(D) shows the result of HLA-DRA RNA expression determined in genetically engineered DC (eDC) using qPCR.

FIG. 4(E) shows the results of GFP-eDC-4 expressions validated by flow cytometry.

FIG. 4(F) shows the results of the test to compare the proliferation of T cells compared to Sp peptide-pulsed DCs.

FIG. 5(A) graphs results to show CD4⁺ and CD8⁺ T cell isolation from peripheral blood mononuclear cells (PBMC).

FIG. 5(B) graphs results to show testing of positive and negative controls generated by treating T cells with or without IL-2 and DCs.

FIG. 6 graphs results of tests to determine clinical CD4⁺ T cell's frequency using various eDC epitopes. FIG. 6 (A) shows results for CD4⁺ T cell frequency induced by eDC epitopes and peptide-pulsed DC (Spep). FIG. 6(B) shows results by flow cytometry for CD4⁺ T cell frequency levels induced by eDC epitopes compared with Spep.

FIG. 7 graphs the results of tests to determine clinical CD8⁺ T cell's frequency using various eDC epitopes. FIG. 7(A) shows CD8⁺ T cell frequency induced by eDC epitopes and peptide-pulsed DC (Spep). FIG. 7(B) shows CD8⁺ T cell frequency levels were significantly higher induced by eDC epitopes compared with Spep.

FIG. 8 graphs results of test on Intracellular IFN-γ⁺ CD4⁺ functional T cells determined using flow cytometry. FIG. 8(A) shows intracellular IFN-γ⁺ CD4⁺ T cell determined by flow cytometry. FIG. 8(B) shows IFN-γ⁺ CD4⁺ T cells representative flow cytometry histogram where eDC-2 showed higher IFN-γ⁺ secreting cells (arrow) compared to other eDC epitopes or Spep.

FIG. 9 graphs results to determine eDC-based IFN-γ, IL-2, and TNF-α secretion from functionally committed CD4⁺ T cells by ELISA. FIG. 9(A) shows that IFN-γ secretion in CD4⁺ T cells showed significantly higher in healthy control (HC), but they are not significantly different with non-treated (NT) T cells. FIG. 9(B) shows that IL-2 secrete substantially higher IFN-γ levels in LongCOV and COV19 samples, and eDC-2-induced this cytokine secretion is significantly different compared to NT CD4⁺ T cells by Dunn's multiple comparisons test. FIG. 9(C) shows that the eDC-2 epitope significantly increased TNF-α (*p<0.05) compared to untreated control samples.

FIG. 10 graphs results to show the viability and safety of eDC epitopes and T cells.

FIG. 11 shows the results of the genetically engineered DC on chronic infection of Long COVID patients, wherein cytomegalovirus (CMV), and BK virus (BKV) proteins were exposed to our DC culture, which may elevate IFN-γ⁺ CD4⁺ T cells in the chronic SARS-CoV-2 (Long COVID) patient samples. FIG. 11(A) shows that CD4⁺ T cell's frequency with BK viral peptides was recognized by these chronically infected T cells and showed a significant difference. FIG. 11(B) shows that BK viral protein-induced CD4⁺ T secreted significantly higher levels of IFN-γ and thus, are functionally committed T cell.

FIG. 12 provides the nucleotide sequence for the full-length Spike protein of SARS-Cov-2_S(ns).

FIG. 13 provides the nucleotide sequence for the S1 region of the Spike protein of SARS-Cov-2.

FIG. 14 provides the nucleotide sequence for the S2 region of the Spike protein of SARS-Cov-2.

FIG. 15 provides the nucleotide sequence for the linker utilized in the constructs depicted in FIG. 4B to link the Spike protein and HLA-DRA.

FIG. 16 provides the nucleotide sequence for human HLA-DRA.

FIG. 17 provides the nucleotide sequence for the SARS-CoV-2 spike protein Signal/Receptor Binding Domain (RBD) utilized in the constructs depicted in FIG. 4B.

FIG. 18 provides the nucleotide sequence for the SARS-CoV-2 spike protein Signal/Receptor Binding Motif (RBM) utilized in the constructs depicted in FIG. 4B.

### DETAILED DESCRIPTION OF THE INVENTION

The present invention will now be described hereinafter with reference to the accompanying drawings and examples, in which embodiments of the invention are shown. This description is not intended to be a detailed catalog of all the different ways the invention may be implemented or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which does not depart from the instant invention. Hence, the following descriptions are intended to illustrate some particular embodiments of the invention and not to exhaustively specify all permutations, combinations, and variations thereof.

Unless otherwise defined, all terms of art, notations, and other Scientific terms or terminology used herein are intended to have the meanings commonly understood by those of skill in the art to which this invention pertains. In some cases, terms with commonly understood meanings are defined herein for clarity and/or for ready reference and understanding, and the inclusion of such definitions herein should not necessarily be construed to mean a substantial difference over what is generally understood in the art.

Commonly understood definitions of molecular biology terms and/or methods and/or protocols can be found in Rieger et al., Glossary of Genetics: Classical and Molecular, 5th edition, Springer-Verlag. New York, 1991; Lewin, Genes V. Oxford University Press: New York, 1994; Sambrook et al., Molecular Cloning, A Laboratory Manual (3d ed. 2001) and Ausubel et al., Current Protocols in Molecular Biology (1994). As appropriate, procedures involving the use of commercially available kits and/or reagents are generally carried out in accordance with manufacturer's guidance.

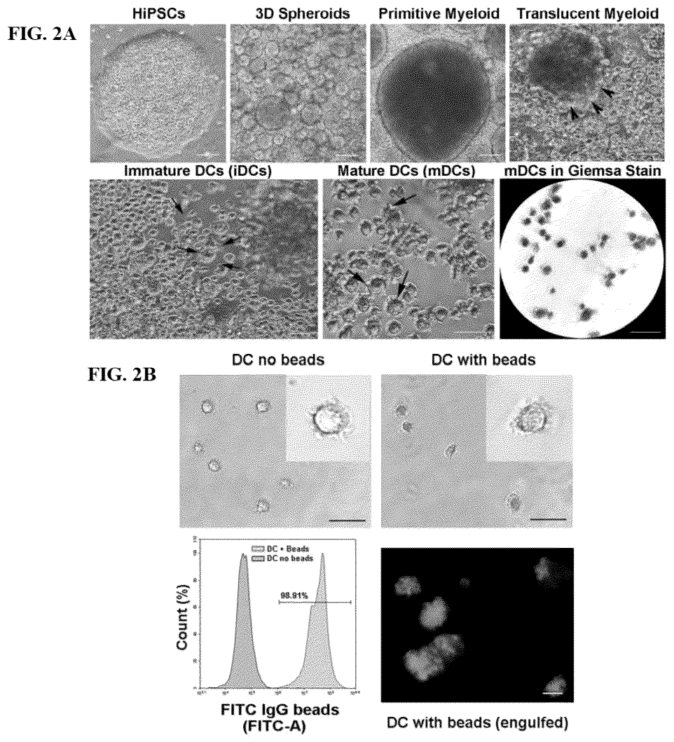

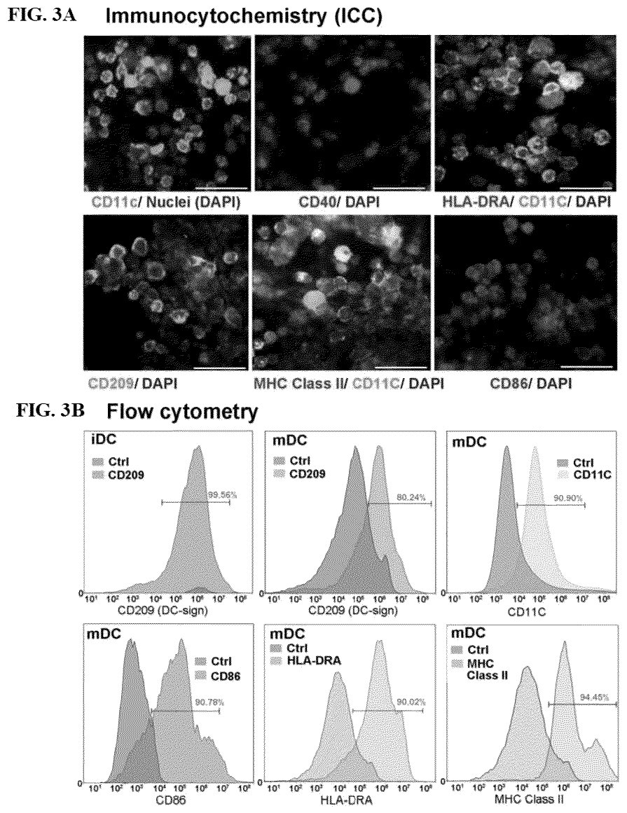

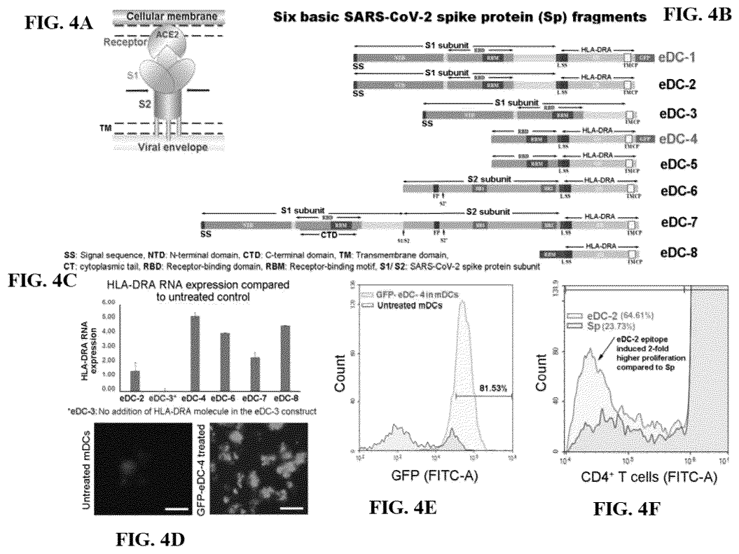

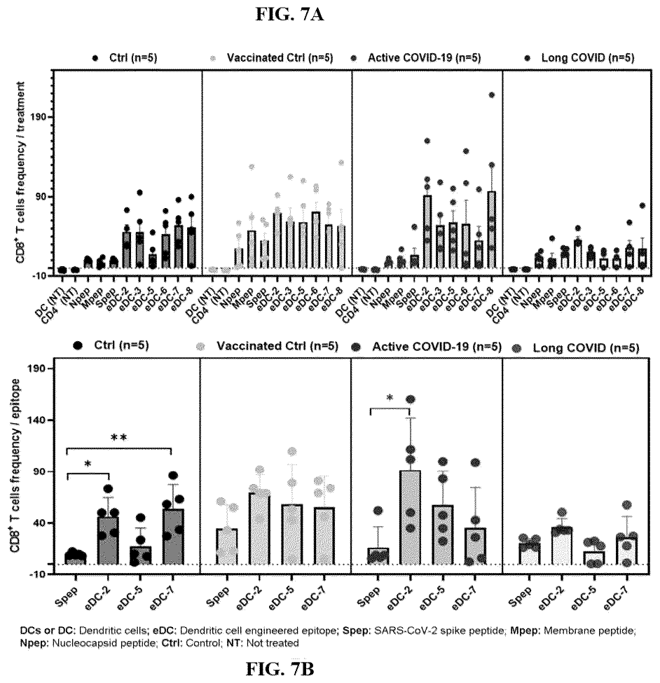

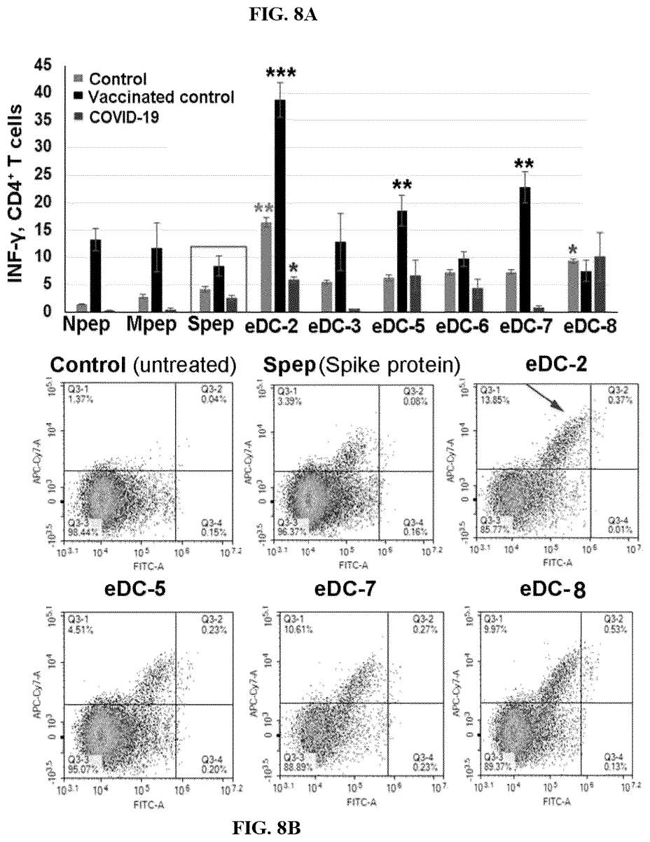

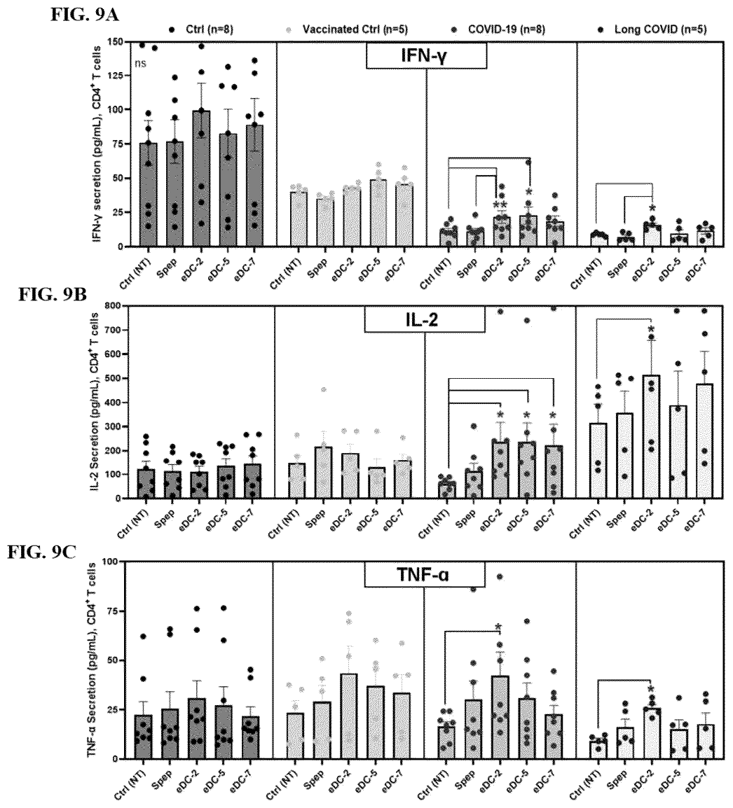

The present invention achieves the following innovative technical and commercial advances:
Developed fully functional DCs from HiPSCs under current good manufacturing practice (cGMP) conditions. DCs are antigen-presenting cells capable of stimulating naive T cells. Thus, DCs are pivotal in generating adaptive immunity and protecting against infections like SARS-CoV-2 or pathogenic infections.
Validated DC's functional activity at >90% by determining the phagocytotic capacity of these DCs. DCs are the immune cells capable of phagocytosing apoptotic cells, with cross-present viral, tumor, and self-antigens to T cells but not macrophages. In this context, we exposed our immature DC (iDC) to fluorescein isothiocyanate (FITC) IgG beads (Cayman Chemical, Ann Arbor, MI) for 24 hours. The inventors determined iDC phagocytose beads>98%, but untreated DC showed no engulfed beads. FITC bead activity was determined by flow cytometry, contrast imaging, and fluorescence microscope (FIG. 2B).
The inventors of this study have characterized DCs with immature and mature DCs markers with >85% efficiency. Inventor also determined immature DCs (iDCs) and mature DCs (mDCs) with established DC markers to validate their functional capability to generate highly efficient engineered DC (eDC) probes to activate T cells more efficiently. We used previously established reported DC markers: CD209⁺ (DC-Sign), CD40⁺, CD11C⁺, CD86⁺, MHC-II⁺, and HLA-DRA⁺ for immunocytochemistry (ICC) and flow cytometry as a previously established method.^{3,14,15} mDCs expressed higher CD40⁺, CD11C⁺, and CD86⁺, as shown in ICC (FIG. 2A). DC-Sign highly expressed iDC (99.5%) but decreased in mature DCs (mDCs) (˜80%), as shown in our flow cytometry histogram (FIG. 2B). In addition, the expression of MHC-II⁺ and HLA-DRA⁺ is vital for the antigen representation in mDCs that tightly regulates T cell activation via T cell receptor (TCR). We found both MHC-II⁺ and HLA-DRA⁺ expressed in our mDCs>90% (FIG. 3B), which validates that our DCs are fully functional and will have the ability to activate T cells.
Verified eDC epitopes with various SARS-CoV-2 constructs with a transduction efficiency≥of 80% using quantitative polymerase chain reaction (qPCR), fluorescence microscope, and flow cytometry (FIGS. 4A-D).
Demonstrated the eDC-linked HLA-DRA epitope's high functionality (p<0.05) by significantly activating and expanding clinical CD4⁺ and CD8⁺ T cells compared to usual peptide-pulsed DCs. In this context, after eDC development using the Sp constructs cultured with CD4⁺ T cells and compared eDC epitopes with Sp-pulsed DC. T cell proliferation increased 2-fold using our eDC epitope compared to Sp-pulsed DC (FIGS. 4E-F). Thus, eDC probe is highly sensitive and has higher specificity to expand T cells as the need for the reduction of SARS-CoV-2, viral-related chronic infections, or Long COVID significantly.
The inventors also introduced our six basic eDC Sp constructs, including spike (Spep), membrane (Mpep), and nucleocapsid (Npep) peptide-pulsed DC to our CD4⁺ and CD8⁺ T cell cultures and incubated them for seven days to determine the frequency of T cells (Division 1-7). It is observed that vaccinated healthy control (VHC), COVID-19 (COV19), and Long COVID (LongCOV) subjects had higher responses with a higher frequency of CD4⁺ and CD8⁺ T cells with our eDC epitopes compared to various SARS-CoV-2 peptide-pulsed DCs such as Spep, Mpep, and Npep which proof our innovative genetically engineered DC-based Sp construct works better than traditionally peptide-pulsed DC. Thus genetically engineered DC is highly sensitive and specific to treat viral or other pathogen infections.
Here also all the eDC epitopes (eDC-2, eDC-3, eDC-5, eDC-6, eDC-7, and eDC-8) were compared with spike protein pulsed DC (Spep) and found that eDC-2, eDC-5, and eDC-7 had a significantly higher response on CD4⁺ T cell frequencies than Spep. More interestingly, the Si subunit of HLA-DRA molecule combination eDC-2 showed significantly higher responses (p<0.05) on activating CD4⁺ T cells in all clinical samples using One-way ANOVA Dunn's multiple comparisons tests (GraphPad Prism, San Diego, CA).
CD8⁺ T cells that are used with the innovative genetically engineered eDC epitopes due to their sustained immunity in patients with chronic viral infection. The inventors also found CD8⁺ T cell response with eDC-2, eDC-5, and eDC-7 increased>2-4 folds depending on the epitopes compared to Spep (FIG. 7A). In addition, using One-way ANOVA Dunn's multiple comparisons tests, the eDC-2 epitope again showed a significantly higher response in CD8⁺ T cell activation compared to Spep in HC (healthy control) and COV19 (COVID-19) samples. LongCOV (Long COVID) still showed a higher T cell response than Spep, even though statistically insignificant may be due to the higher exhaustion of CD8⁺ T cells in chronic infection. Our overall data indicates that eDC-2 is a highly efficient probe elevating CD4⁺ and CD8⁺ T cell frequencies in all clinical samples regardless of T cell exhaustion. Thus, eDC-2 is the innovative genetically engineered DC that can produce highly efficient T cell therapy against viral infections.
Activation of T cells results in intracellular expression and secretion of cytokines such as IFN-γ, established in various clinical studies.^{16,17,18} Thus, inventors examined whether activated T cells by eDC epitopes can secrete intracellular IFN-γ, which led to measuring intracellular IFN-γ⁺ using anti-IFN-γ antibodies (BD Biosciences) with flow cytometry. It is observed that eDC epitopes induced higher levels of IFN-γ expression, specifically with the eDC-2, eDC-5, eDC-7, and eDC-8 epitopes in healthy control (HC), VHC, and COV19 samples, compared to the spike protein pulsed DC (Spep). Thus all the eDC epitopes were compared to the Spep to perform a statistically significant comparison (*p<0.05 and **p<0.01).
The inventors also measured IFN-γ, IL-2, and TNF-α in CD4⁺ T cell media and found all cytokines increased in SARS-CoV-2 patients as reported publications.^{16,17,18} The above mentioned cytokines were measured using ELISA kits from StemCell Technologies or BioLegends (San Diego, CA). The results showed that IFN-γ, IL-2, and TNF-α were significantly elevated in the COV19 and LongCOV patient samples compared to HC and VHC. Interestingly, IL-2 substantially increased in all the clinical samples in higher levels (>200 pg/mL) compared to IFN-γ and TNF-α (<100 pg/mL). eDC epitopes-based IFN-γ, IL-2, and TNF-α secretion was compared with Spep and found that most of our eDC epitopes (eDC-2, eDC-5, eDC-7, and eDC-8) secrete significantly higher levels of cytokines in COV19, and LongCOV compared to Spep. Moreover, Spep had no significant difference in secreting these cytokines compared to non-treated control samples, which validates that our eDC epitopes are superior to any peptide-pulsed DC like Spep usually studied in traditional T cell proliferation studies.^{16,17,18} eDC-2 results consistently showed significantly higher levels of IFN-γ secretion (p<0.05) in both cellular and media fractions in CD4⁺ T cells (FIGS. 8-9). Overall results indicated that eDC-2 is highly sensitive in secreting increased levels of IFN-γ (>25 pg/mL), IL-2 (>200 pg/mL), and TNF-α (>35 pg/mL) compared to any other eDC epitopes and Spep, which validates its use for genetically engineered innovative eDC-2 probe for efficient T cell therapy.
The innovative eDC epitope is not limited to SARS-CoV-2 but can be expanded to other viral pathogens, such as CMV and BKV. Furthermore, the inventors found robust and significant frequencies of CMV and BKV protein-specific CD4⁺ T cells that produced increased levels of IFN-γ response (p<0.05). Thus, SARS-CoV-2, CMV, and BKV-based eDC epitopes can be used simultaneously to treat patients with LongCOV and transplantation-related multiple viral infections and pathogen-related chronic or degenerative disorders efficiently.
DC and T cell viability and safety were essential concerns in developing this T cell-based therapy for patients. Therefore, after the transduction of DC with various genetically modified epitopes and the addition of T cells, the inventors examined the mixed culture viability compared to untreated cells using the 3-(4,5-dimethylthiazol-2-yl)-2,5-diphenyltetrazolium bromide (MTT) assay. MTT is a yellow dye that cellular enzymes reduce to the blue product formazan if cells are viable.^{19,20} The viability of the mixed culture of all DC epitopes and T cells was >80%. Moreover, here IL-2 was added in mixed culture and observed>7 cell division within seven days of culture, typically observed in T cell proliferation. In this context, DC transduction with various epitopes or T cell isolation processes did not cause toxicity. Thus, our transduction and isolation processes for the DC and T cells were highly viable and safe.

### Definitions

Unless otherwise defined, all technical and scientific terms used herein have the same meaning as commonly understood by one of ordinary skill in the art to which this invention belongs. The terminology used in the description of the invention herein is to describe particular embodiments only and is not intended to be limiting to the invention.

All publications, patent applications, patents, and other references cited herein are incorporated by reference in their entirety for the teachings relevant to the sentence and paragraph in which the reference is presented.

As used in the description of the invention and the appended claims, the singular forms “a,” “an,” and “the” are intended to include the plural forms as well unless the context clearly indicates otherwise.

The term “HiPSCs” refers to human induced pluripotent stem cells with similar phenotypical and genotypical characteristics as human embryonic stem cells (hESCs) or human pluripotent stem cells (HPSCs). Human somatic cells have been reprogrammed directly to pluripotency by ectopic expression of four transcription factors (Oct4, Sox2, Klf4, and Myc) to yield HiPSCs. HiPSCs have self-renewing capabilities similar to hESCs and can undergo three germ layers, producing all the germ layer cells with appropriate growth factors, such as mesodermal lineage-derived dendritic cells or blood-related cells and tissues.

The term “DC” or “DCs” is a singular or plural form used to express due to their use in a certain cell culture environment or to perform a specific assay. DCs are dendritic cells, the type of phagocytes, and the antigen-presenting cell that can be found in tissues such as skin, and it boosts immune response by showing antigens on its surface to others of the immune system such as T cells. We have generated iDC and mDC, which are immature and mature DC, respectively. iDC usually patrols the organism at an immature stage to detect the presence of pathogens. After being activated by a foreign pathogen protein, it becomes active and mature. Mature DCs express increased levels of fascin-1, an action-bundling protein, that enhances their migration, and they tend to move toward lymph nodes using the active movement of dendrites at a higher speed than the immature cells.

The term eDC is used to refer to a genetically engineered DC or engineered DC. We have integrated a viral construct of spike protein (Sp) of SARS-CoV-2 into DC's genome which we called eDC epitopes. These novel eDC epitopes can be used to expand SARS-CoV-2 protective T cells and treat patients with SARS-CoV-2 chronic infection or Long COVID.

Long COVID is referred to as LongCOV or long-haul COVID, post-acute COVID-19, long-term effects of COVID, or chronic COVID, which involves a variety of new, returning, or ongoing symptoms that people experience more than four weeks after getting COVID-19.

HLA-DR or HLA-DRA is human leukocyte antigen—DR or DRA isotype (HLA-DR or HLA-DRA) covalently linked to the protein (our case Sp), causes DCs to generate robust CD4⁺ and CD8⁺ T cells in patients regardless of race and ethnicity since HLA-DRA is found in 98% of the human population.^{3,4} HLA is a type of molecule found on the surface of most cells in the body. Human leukocyte antigens play an important part in the body's immune response to foreign substances. They make up a person's tissue type, which varies from person to person. Human leukocyte antigen tests are done before a donor stem cell or organ transplant to find out if tissues match between the donor and the person receiving the transplant. Also called HLA and human lymphocyte antigen.

There are various SARS-CoV-2 proteins with simplified renames, such as spike protein (Sp), membrane protein (M), and nucleocapsid (N) protein. These important proteins can be expressed in our DCs, producing these Sp, M, and N protein-specific CD4⁺ and CD8⁺ T cells ex vivo to treat patients with post-COVID infections or others.

The present invention is also directed to engineered DCs with other viral proteins. Thus, the DCs of the invention are not limited to SARS-CoV-2 but also encompass DCs to be used for other virus, pathogens or infectious agents, including cytomegalovirus or the polyomavirus family beta polyomavirus (BK) virus for potential delivery back into the patients to reduce or stop the risk of transplant-related infections, chronic infection, and Long COVID. Cytomegalovirus (CMV) is a common infection caused by herpes virus. CMV is the virus that usually causes infection in pregnant women and populations suffering from chronic infection, especially from immunosuppression. BK virus (BKV) is estimated to cause a progressive kidney transplant injury in 1-10% of renal transplant recipients. BK virus disease screening, early treatment benefit, and long-term transplant survival, which we can do with our genetically engineered novel DC by developing BK-specific eDCs.

Chronic infections, immunosuppressive disorders, and older populations: Chronic infection may not necessarily cause symptoms, may still be active, and may spread to others. Chronic infections may last for years, such as influenza, post-COVID infections, hepatitis B, tuberculosis, etc. People are said to be immunosuppressed when they have an immunodeficiency disorder due to medicines that weaken the immune system: for example, Lupus, multiple sclerosis, psoriasis, and rheumatoid arthritis. Older populations referred to as aged 65 or over usually often suffer T cell exhaustion due to impaired dendritic cell functions, which our novel eDC system can treat.

DCs are “Phagocytosed” cells, and we verified their functional activities by performing a phagocytosis assay. Thus, we exposed our immature DC (iDC) to fluorescein isothiocyanate (FITC) IgG beads (Cayman Chemical, Ann Arbor, MI) for 24 hours. We found iDC phagocytose beads>98%, but untreated DC showed no engulfed beads. Immunoglobulin G (IgG) is the most common antibody. It's in blood and other body fluids and protects against bacterial and viral infections. IgG can take time to form after an infection or immunization.

Phagocytosis is the process by which a cell uses its plasma membrane to engulf a large particle, giving rise to an internal compartment called the phagosome. It is one type of endocytosis. A cell that performs phagocytosis is called a phagocyte.

We referred to DC markers means are the proteins specifically expressed in DCs. They allow for the detection and identification of distinct DC types by using different techniques. Our system used immunocytochemistry (ICC) and flow cytometry. We have determined immature DCs (iDCs) and mature DCs (mDCs) with established DC markers to validate the functional capability of our DC for their immunoregulatory functions. There is an example for various proteins expressed in our DCs, such as CD209⁺ (DC-Sign), CD40⁺, CD1c⁺, CD11C⁺, CD86⁺, MHC Class-II⁺ (MHC-II⁺) and HLA-DRA⁺ for immunocytochemistry (ICC) and flow cytometry as a previously established method developed.^{3,14,15}

Giemsa stain is used here to visualize DC and its long processes and long extensions of dendrites. Giemsa stain is one of the best-known histological stains, coloring the nuclei dark blue and the cytoplasm blue to pink, according to the acidity of the cytoplasmic contents.

MHC Class II or MHC-II⁺ refers to major histocompatibility complex (MHC) class II molecules that present processed antigens derived primarily from exogenous sources to CD4⁺ T lymphocytes or T cells. MHC class II molecules are critical for initiating the antigen-specific immune response.

Lymphocytes or T cells are a type of immune cell that is made in the bone marrow and is found in the blood and lymph tissue. There are mainly two types of lymphocytes: B and T. B lymphocytes make antibodies, and T lymphocytes help kill tumor cells and help control immune responses.

CD4⁺ or CD8⁺ T lymphocytes or cells refer to a type of white blood cell in your immune system. CD4⁺ T cells lead the fight against infections. CD8⁺ T cells can kill cancer cells and other invaders. If a patient has HIV, the CD4⁺ T cell count may be low.

eDC epitopes refer to genetically engineered or modified DC with various viral constructs of the spike protein of SARS-CoV-2. We have referred to eDC-1 epitopes which contain the full length of the spike protein of SARS-CoV-2 with S1 and S2 subunits and green fluorescent protein (GFP), but eDC-2 epitopes also contain the full length of the spike protein of SARS-CoV-2, but there is no GFP. eDC-3 epitopes only contain the Si subunit of spike protein, but eDC-6 contains only the S2 subunit of the spike protein of SARS-CoV-2. eDC-4 contains only the receptor-binding domain (RBD) and receptor binding motif (RBM) of the spike protein of SARS-CoV-2 with GFP protein, but eDC-5 contains all the same as eDC-4 but no GFP. eDC-7 contains the full length of the spike protein of SARS-CoV-2 with subunits S1 and S2, but no linked molecule HLA-DRA added to the Sp construct like all other epitopes. eDC-8 contains no spike protein of SARS-CoV-2, only containing the HLA-DRA construct used here as a negative control.

Here referred “peptide pulsed DC” which means DC pulsed with antigen (protein), is a versatile approach to generate T cell immunotherapy or vaccines. DCs are the most effective type of antigen-presenting cells and can stimulate naive T cells to initiate a primary immune response. We introduced our six basic eDC Sp constructs, including spike peptide (Spep), membrane peptide (Mpep), and nucleocapsid peptide (Npep) of SARS-CoV-2 used in DC. Here pulsed refers to peptide added to the DC culture. Then DC can present such peptide (antigen) to the cell surface to activate T cells to secrete cytokine and control inflammation or prevent disease.

In this invention, eDC epitopes were used against some clinical samples referred to as healthy control (HC), vaccinated healthy control (VHC), COVID-19 (COV19), and Long COVID (LongCOV) subjects. VHC, COV19, and LongCOV were all exposed to SARS-CoV-2 protein or viruses and had higher responses with a higher frequency of CD4⁺ and CD8⁺ T cells with our eDC epitopes compared to various SARS-CoV-2 peptide-pulsed DCs (Spep).

The reference graph estimated T cell frequencies for common cell types found in humans, mice, rat blood, spleen, bone marrow, and thymus. Knowing the frequency of cells in our flow assay is vital to acquire sufficient cells.

ELISA Kit (enzyme-linked immunosorbent assay) is the most widely used enzyme immunoassay technology. Enzyme-linked immunosorbent assay (ELISA), also known as an enzyme immunoassay (EIA), is a biochemical technique used mainly in immunology to detect the presence of an antibody or an antigen in a sample. In our experiment, we used ELISA to detect interferon-gamma (IFN-γ), tumor necrosis factor-alpha (TNF-α), and interleukin (IL)-2 cytokines in our T cells which confirmed that our T cells are fully functional and produce cytokines. IFN-γ, TNF-α, and IL-2 are pro-inflammatory cytokines. Cytokines are signaling proteins that help control inflammation in our body. When the body is inflamed with foreign invaders, T cells secrete some cytokines to control inflammation.

Intracellular IFN-γ is located or occurring within cell or cells. In our case, we are interested in determining located within the T cells.

Genetically engineered DC (eDe) quality was determined to verify its novelty use in high throughput study. In this context, we have determined the viability and stability using MTT [3-(4,5-dimethylthiazol-2-yl)-2,5-diphenyitetrazoliumbromide)] assay. MTT assay is used to measure cellular metabolic activity as an indicator of cell viability, proliferation, and cytotoxicity that confirmed our modified DC in T cell didn't cause any toxicity or not reduced cellular viability. These systems may be toxic if cell viability goes ≤70%. But our eDC overall maintained cell viability≥80%. Thus our eDC is completely safe, and no toxicity is observed in eDC and T cell mix culture.

NT or UT is often used to compare control against treated samples where NT abbreviates not treated control. UT is also used for the same purposes as NT, which translates untreated control compared to treated sample example. I cells are treated with IL-2 (factor or compounds or materials), but untreated or not treated T cells do not use IL-2 or any other factor, compound, or materials.

Dunn's multiple comparisons test is a statistical test that compares the difference in the sum of ranks between two columns with the expected average difference (based on the number of groups and their size). For each pair of columns, GraphPrism (San Diego, CA) reports the P value as >0.05 (not significant) but <0.05 significant such as *<0.05, **<0.01,***<0.001, or ****<0.001.

The term “pathogenic protein” refers to a protein expressed on the surface of a pathogen, such as a virus, bacteria or other pathogenic or infectious agent.

The term “degenerative protein” refers to a protein modifications that have been strongly implicated in the molecular pathogenesis of several age-related diseases affecting the cardiovascular system (CVS) and central nervous system (CNS), including atherosclerosis, heart disease, dementia syndromes, and stroke. Examples of degenerative proteins like amyloid beta and tau for Alzheimer's disease, and alpha-synuclein for Parkinson's disease.

The term “fragment”, as applied to polynucleotide sequences, refers to a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of oligonucleotides ranging in length from at least 6, 8, 9, 10, 12, 15, 18, 20, 21, 22, 23, 24, 25, 30, 39, 40, 42, 45, 48, 50, 51,54, 57, 60, 63, 66, 70, 78,80, 90, 100,105, 120, 135, 150, 200, 300, 500, 720, 900, 1000, 1500, 2000, 3000, 4000, 5000, or more consecutive nucleotides of a nucleic acid according to the invention.

As used herein, an “isolated nucleic acid fragment” refers to a polymer of RNA or DNA that is single- or double stranded, optionally containing synthetic, non-natural or altered nucleotide bases. An isolated nucleic acid fragment in the form of a polymer of DNA may be comprised of one or more segments of cDNA, genomic DNA or synthetic DNA.

A DNA “coding sequence” refers to a double-stranded DNA sequence that encodes a polypeptide and can be transcribed and translated into a polypeptide in a cell in vitro or in vivo when placed under the control of suitable regulatory sequences.

“Suitable regulatory sequences’ refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites, and stem-loop structures. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

“Open reading frame’ is abbreviated ORF and refers to a length of nucleic acid sequence, either DNA, cDNA or RNA, that comprises a translation start signal or initiation codon, such as an ATG or AUG, and a termination codon and can be potentially translated into a polypeptide sequence. The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription.

A “vector” refers to any vehicle for the cloning of and/or transfer of a nucleic acid into a host cell. A vector may be a replicon to which another DNA segment may be attached. So as to bring about the replication of the attached segment. A “replicon refers to any genetic element (e.g., plasmid, phage, cosmid, chromosome, virus) that functions as an autonomous unit of DNA replication in vivo, i.e., capable of replication under its own control. The term “vector” includes both viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacterio phages such as lambda derivatives, or plasmids such as pBR322 or puC plasmid derivatives, or the Bluescript vector. Another example of vectors that are useful in the invention is the UltraVector™ Production System (Intrexon Corp., Blacksburg, Va.) as described in WO 2007/038276. For example, the insertion of the DNA fragments corre sponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal Subjects. Viral vectors that can be used include, but are not limited to, retrovirus, adeno associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

The term “plasmid’ refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. A “cloning vector” refers to a “replicon, which is a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (”shuttle vector). Cloning vectors may comprise one or more sequences that can be used for selection of cells comprising the vector and/or one or more multiple cloning sites for insertion of sequences of interest.

Vectors may be introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., J. Biol. Chem. 267:963 (1992); Wu et al., J. Biol. Chem. 263: 14621 (1988); and Hartmut et al., Canadian Patent Application No. 2,012,311).

The term “transfection” refers to the uptake of exogenous or heterologous RNA or DNA by a cell. A cell has been “transfected by exogenous or heterologous RNA or DNA when such RNA or DNA has been introduced inside the cell. A cell has been “transformed by exogenous or heterologous RNA or DNA when the transfected RNA or DNA effects a phenotypic change. The transforming RNA or DNA can be integrated (covalently linked) into chromosomal DNA making up the genome of the cell.

The term “transduction” refers to the transfer of genetic material, RNA or DNA, from one cell to another by means of a virus. Dendritic cells, such as those in the present invention, can be genetically engineered via transduction by means of various vectors that are per se known in the art, including viral vectors, and lentiviral vectors. 21 Zsarei et al, BASIC AND CLINICAL IMMUNOLOGY1 VOLUME 109, ISSUE 6, P988-994, JUNE 2002

“Transformation” refers to the transfer of a nucleic acid fragment into the genome of a host organism, resulting in genetically stable inheritance. Host organisms containing the transformed nucleic acid fragments are referred to as “transgenic’ or “recombinant’ or “transformed’ organisms. In addition, the recombinant vector comprising a poly nucleotide according to the invention may include one or more origins for replication in the cellular hosts in which their amplification or their expression is sought, markers or selectable markers.

The term “operably linked’ refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein refers to the transcription and stable accumulation of sense (mRNA) or antisense RNA derived from a nucleic acid or polynucleotide. Expression may also refer to translation of mRNA into a protein or polypeptide.

The terms “cassette,” “expression cassette’ and “gene expression cassette’ refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation.

“Transformation cassette’ refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

The term “fragment”, as applied to a polypeptide, refers to a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide, according to the invention may have a length of at least 2, 3, 4, 5, 6, 8, 10, 13, 14, 15, 16, 17, 18, 19, 20, 21, 22, 25, 26, 30, 35, 40, 45, 50, 100, 200, 240, or 300 or more amino acids.

The term “viral surface proteins” and “viral membrane proteins” refer to proteins that can be found in a viral envelope or cell membrane that are capable of stimulating an effective T cell response to activate and cause the proliferation of activated T cells by the immune system.

### DETAILED DESCRIPTION OF DRAWINGS

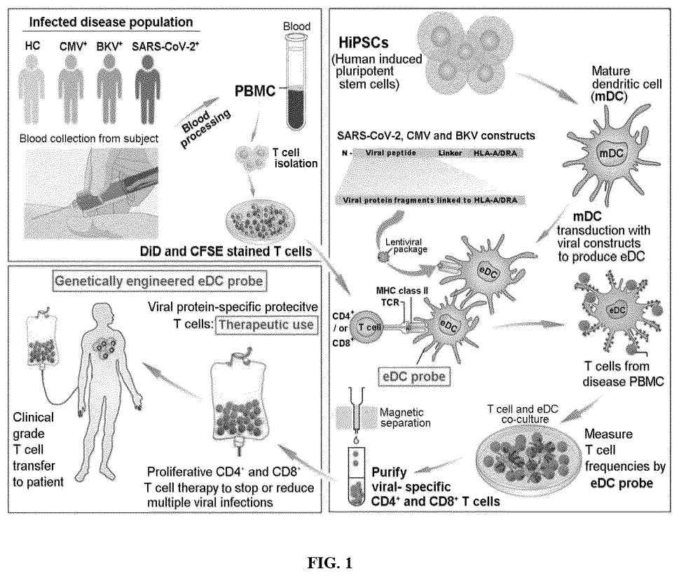

FIG. 1. Stepwise generation of genetically engineered DC probe from HiPSCs. DC-COV19 activated a multi-epitope-based broad spectrum of SARS-CoV-2, and other viral-specific T cells will be used to develop a next-generation theranostic product to treat current and newly emerging SARS-CoV-2 and transplant-related viral infection.

FIG. 2. Generation of naive DCs from HiPSCs. (A) Dissociated HiPSCs were treated with mesodermal growth factors to generate 3D spheroids and further generation of myeloid and immature dendritic cells (iDCs) with protrusion and veils type morphology. We treated iDCs with maturation factors to generate mature DCs (mDCs) with long dendrites. Arrowhead showed translucent DC, which time-dependently released iDC, but the arrow showed iDC with protrusion ad veils type characteristics. Arrow also showed long processes in mDC, and Gimesa staining showed long dendrites. (B) We have tested DC's phagocytic activity with FITC-IgG beads for 24 h, and DCs successfully engulfed beads>90%, which validates our DCs functionality. Scale bars: 70-300 μm.

FIG. 3. DC characterization by ICC and flow cytometry. DC's functional activities and morphological characteristics were determined and validated with immature DCs (iDCs) and mature DCs (mDCs) marker expressions (CD209⁺, CD40⁺, CD11C⁺, CD86⁺, HLA-DRA⁺, and MHC Class II⁺) by (A) ICC and (B) flow cytometry. HLA-DRA⁺ and MHC Class II⁺ expression, including other mature DCs markers, showed >90%. Scale bars: 70-300 μm.

FIG. 4. Fabrication of genetically engineered DCs (eDCs). (A) SARS-CoV-2 spike protein (Sp) structure to enter human cellular levels. (B) Eight Sp-based epitopes where two epitopes were labeled with GFP. Thus, six basic Sp constructs which are extensively used in DC transduction. (C) GFP-eDC-4 constructs were added to mature dendritic cells (mDCs) and found to be highly expressed in mDCs compared to untreated cells. (D) HLA-DRA RNA expressions were determined in genetically engineered DC (eDC) using qPCR and found Sp constructs significantly entered in the mDCs genome. It is important to note that eDC-3 integrated construct is similar to the Sp construct of eDC-2, except no addition of the HLA-DRA molecule validates no extra HLA-DRA RNA expression compared to eDC-2 or other eDCs. (E) GFP-eDC-4 expressions were also validated by flow cytometry which expressed ≥80% compared to untreated mDCs. (F) eDC-2 epitopes showed a 2-fold higher proliferation of T cells compared to Sp peptide-pulsed DCs, which validates our eDCs have higher sensitivity and specificity to activate T cells compared to traditional peptide-pulsed DCs. Scale bars: 100 μm.

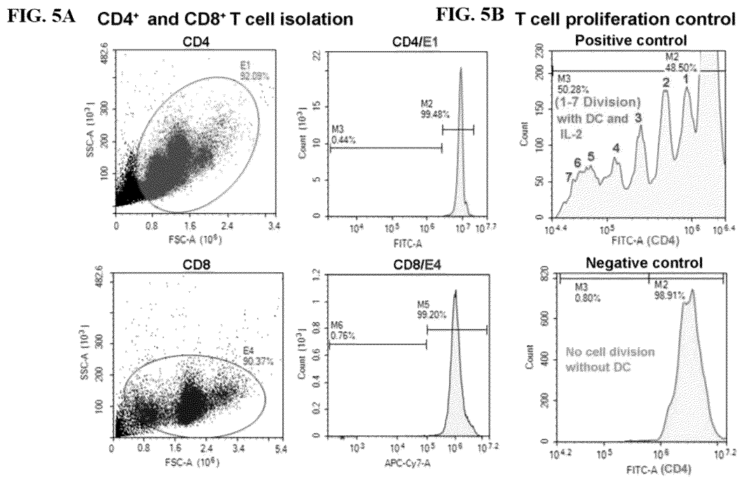

FIG. 5. Validation of CD4⁺ and CD8⁺ T cell purity. (A) CD4⁺ and CD8⁺ T cell isolation from peripheral blood mononuclear cells (PBMC) were >90% on average, validating higher T cell purity. (B) Positive and negative control were generated by treating T cells with or without IL-2 and DCs. Cell divisions were calculated by using these controls.

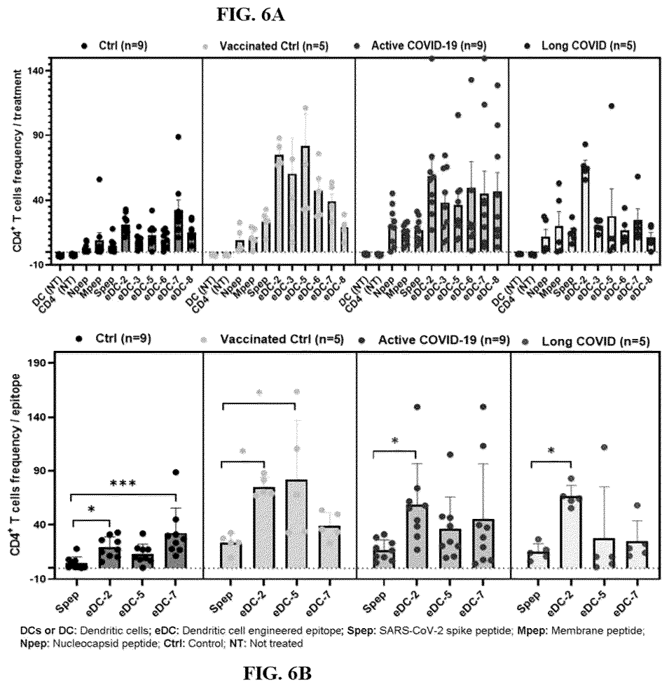

FIG. 6. Determine clinical CD4⁺ T cell's frequency using various eDC epitopes. (A) CD4⁺ T cell's frequency induced by eDC epitopes and peptide-pulsed DC (Spep) was determined by counting 1-7 cell division by flow cytometry. (B) CD4⁺ T cell frequency levels were significantly higher induced by eDC epitopes compared with Spep (*p<0.05, ***p<0.001) by Dunn's multiple comparisons tests. eDC-2 epitope showed higher CD4⁺ T cell frequency than other eDC epitopes. Thus eDC-2 is the novel DC probe for activating T cells for new and existing SARS-CoV-2.

FIG. 7. Determine clinical CD8⁺ T cell's frequency using various eDC epitopes. Clinical CD8⁺ T cells were cultured with the eDC epitopes and peptide-pulsed DC sample. We found CD8⁺ T cell frequencies with our epitopes substantially decreased in LongCOV subjects but increased in the VHC and COV19 samples compared to the HC. (A) CD8⁺ T cell's frequency induced by eDC epitopes and peptide-pulsed DC (Spep) was determined by counting 1-7 cell division by flow cytometry. (B) CD8⁺ T cell frequency levels were significantly higher induced by eDC epitopes compared with Spep (*p<0.05, ***p<0.001) by Dunn's multiple comparisons tests.

FIG. 8. Intracellular IFN-γ⁺ CD4⁺ functional T cells determined using flow cytometry. (A) Intracellular IFN-γ⁺ CD4⁺ T cell determined by flow cytometry. We have compared eDC epitopes with spike peptide-pulsed DC (Spep, red box) and found eDC-2 epitope significantly higher (p<0.05) expressed IFN-γ⁺ cells. (B) IFN-γ⁺ CD4⁺ T cells representative flow cytometry histogram where eDC-2 showed higher IFN-γ⁺ secreting cells (arrow) compared to other eDC epitopes or Spep. *p<0.05, **p<0.01, ***p<0.001 denote significant comparing cases (control, vaccinated control, COVID-19) with matching color-coded bars and stars (*). Intracellular IFN-γ expression where eDC-2 epitopes showed higher IFN-γ secretion than any eDC epitopes or peptide-pulsed DC. Overall results indicated that the eDC-2 epitope is the innovating engineered DC for T cell therapy for the chronic infection population more efficiently.

FIG. 9. eDC-based IFN-γ, IL-2, and TNF-α secretion from functionally committed CD4⁺ T cells by ELISA. IFN-γ, IL-2, and TNF-α secretion was determined in CD4⁺ T cell media by ELISA. (A) IFN-γ secretion in CD4⁺ T cells showed significantly higher in healthy control (HC), but they are not significantly different with non-treated (NT) T cells. HC usually proliferates T cells at higher levels with the spontaneous secretion of pro-inflammatory cytokines, as observed in our samples. (B) IL-2 secrete substantially higher IFN-γ levels in LongCOV and COV19 samples, and eDC-2-induced this cytokine secretion is significantly (*p<0.05, ***p<0.001) different compared to NT CD4⁺ T cells by Dunn's multiple comparisons test, providing insight that they have active or chronic infection. (C) Here eDC-2 epitope significantly increased TNF-α (*p<0.05) compared to untreated control samples. Overall results indicate that our eDC-2 is the most potent genetically engineered DC epitope for activating functionally committed CD4⁺ T cells, which can protect against SARS-CoV-2 and LongCOV infections with higher sensitivity and specificity.

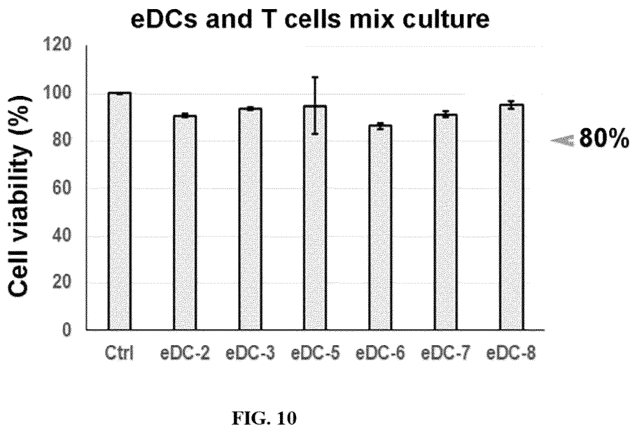

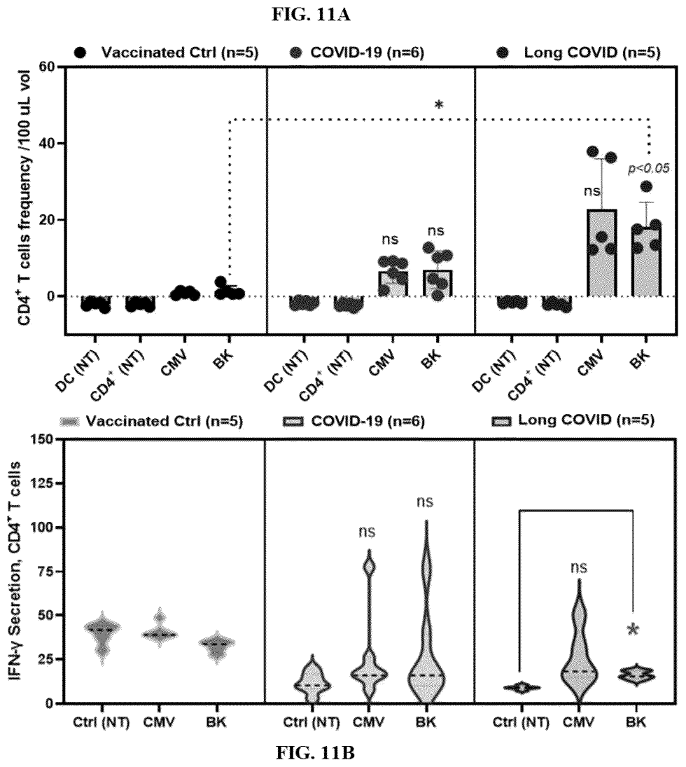

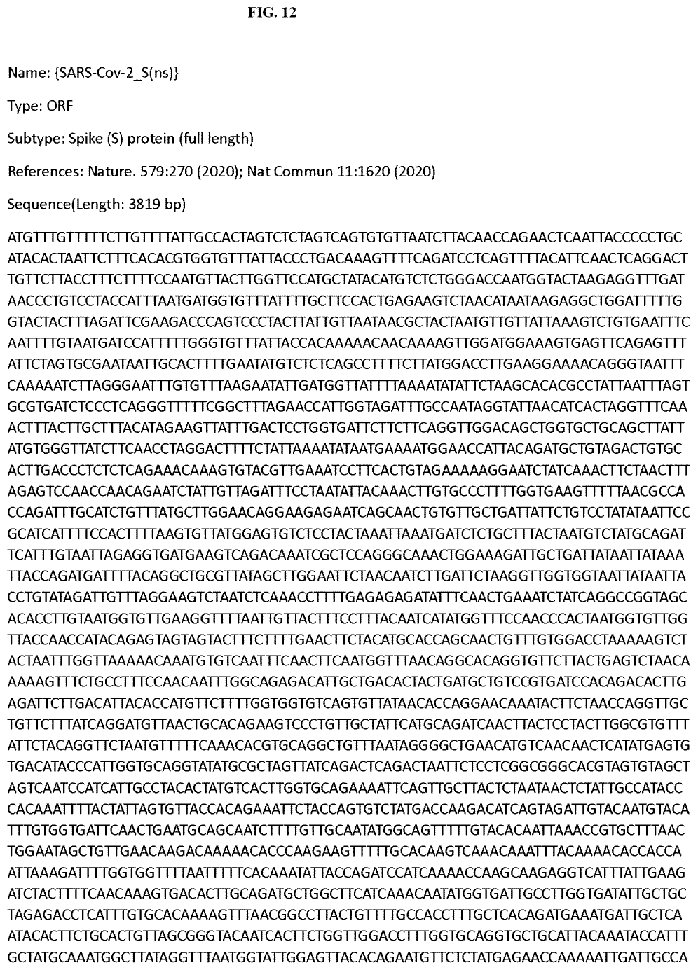

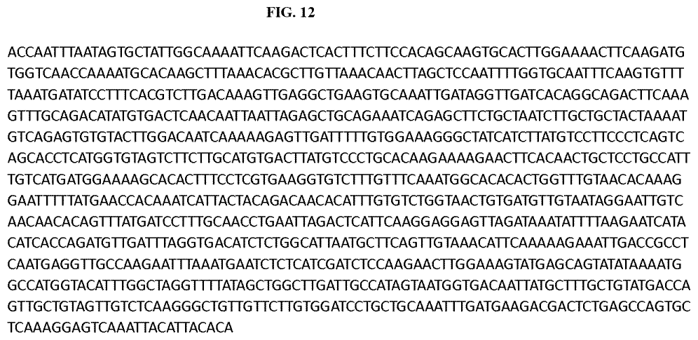

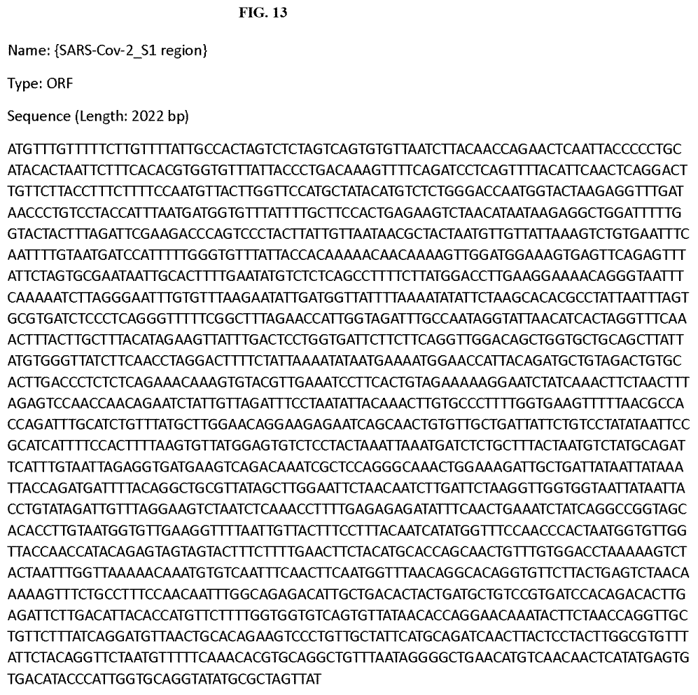

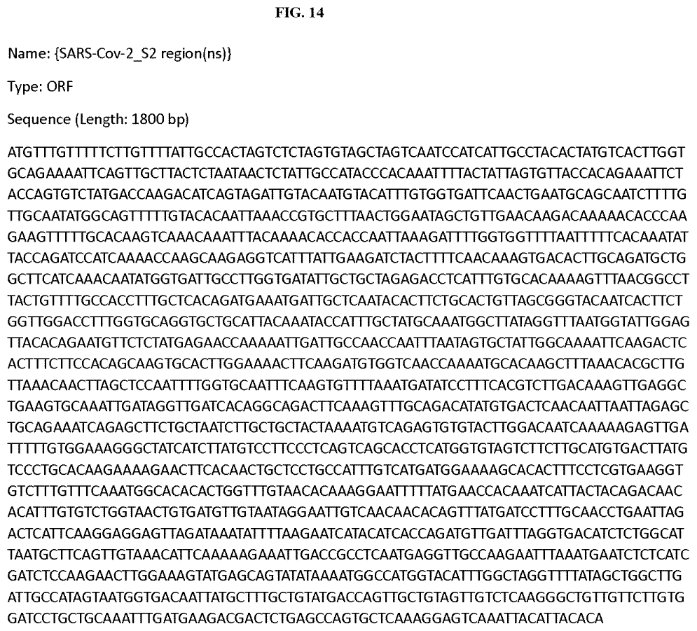

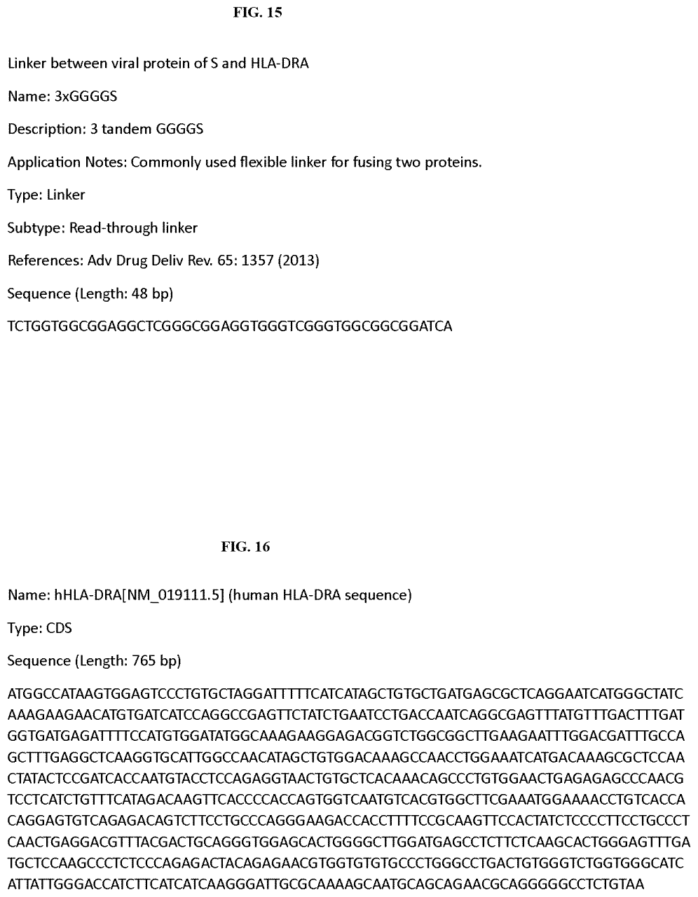

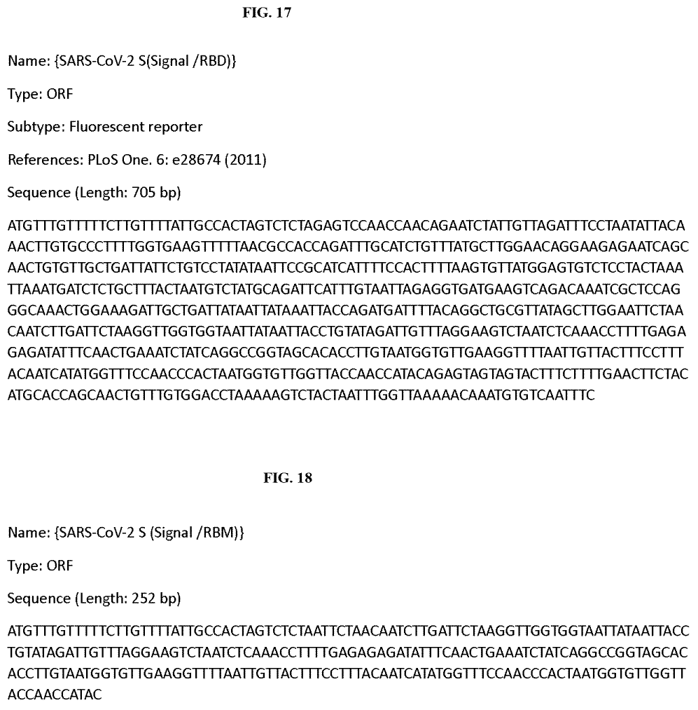

FIG. 10. Viability and safety of eDC epitopes and T cells. DC and T cell viability and safety were essential concerns in developing this T cell-based therapy for patients. Therefore, after the transduction of DC with various epitopes and the addition of T cells, we examined the mixed culture viability compared to untreated cells using the 3-(4,5-dimethylthiazol-2-yl)-2,5-diphenyltetrazolium bromide (MTT) assay. We examined T cell viability when mixed with eDCs culture compared to untreated cells using MTT assay. Viability was >80% in all cases.

FIG. 11. Genetically engineered DC on chronic infection of Long COVID patients. To understand DC-based T cell theragnostic potential, we exposed cytomegalovirus (CMV), and BK virus (BKV) proteins to our DC culture, which may elevate IFN-γ⁺ CD4⁺ T cells in the chronic SARS-CoV-2 (Long COVID) patient samples. Thus, inventors have treated Long COVID (chronic SARS-CoV-2 samples where three samples were diabetes and chronic infection usually linked to kidney infection such as BK viral infection. (A) CD4⁺ T cell's frequency with BK viral peptides was recognized by these chronically infected T cells and showed a significant difference. (B) We have also determined whether BK viral protein-induced CD4⁺ T cells are functionally committed T cells and found they secreted significantly higher levels of IFN-γ. Thus, BK viral protein-induced CD4⁺ T cells are functionally committed T cells to the future prevention of transplantation-related infections (*p<0.05), which provides novel insight into our technology.

### Preparation of Genetically Engineered DCs

The genetically engineered DCs of the invention are prepared by the following general procedure:

Step 1: Generation of 3D spheroid: Culture HiPSCs in a spheroid growth medium, such as a stabilized, serum-free cell culture medium for the feeder-free maintenance and expansion of human embryonic stem cells (hES) and human induced pluripotent stem cells (HiPSCs), wherein the spheroid culture medium comprises factors comprising mesodermal growth factors, consisting of human recombinant (rh) bone morphogenetic protein 4 (BMP4), rh vascular endothelial growth factor (VEGF), rh stem cell factor (SCF), and rh granulocyte-macrophage colony-stimulating factor (GM-CSF). The thus produced 3D spheroid cells can be observed and selected from other cells. A spheroid is simply an ellipsoid that approximates a sphere. Spheroids are self-organized, simple, widely used multicellular 3D models that form round 3D aggregate, which can be able to use for differentiating cells of interest with appropriate growth factors.

Step 2: Generation of myeloid cells: The DC culture medium comprising the 3D spheroids is supplemented with additional factors comprising rh BMP4, rh VEGF, rh SCF, and rh GM-CSF.

Step 3: Generation of immature DCs (iDCs): Remove BMP4, VEGF, and SCF from the culture and add IL-4 to the DC culture medium.

Step 4: Generation of mature DCs (mDCs): The thus produced iDCs are removed, such as by means of a cell strainer to remove aggregates. The iDCs are then cultured in a DC culture medium comprising rhGM-CSF, rhlL-4, rhTNF-α, rhIFN-γ, prostaglandin E2 (PGE2), and rhIL-1β to generate mDCs.

Step 5: Genetically engineer DCs: A suitable vector, such as a viral vector, such as lentivirus vector comprising the fusion construct of the virus protein/antigen (such as SARS-CoV2 spike protein (Sp) (full length or fragment such as subunits S1 and/or S2)) and HLA-DRA prepared by VectorBuilder^{22,23,24} are used to transfect mDC to produce the desired eDCs.

### Use of Genetically Engineered DCs

Step 6: T cell activation with eDC epitopes: The eDCs are co-cultured with T cells to produce viral-specific CD4⁺ and CD8⁺ T cells.

T cells can be obtained from peripheral blood mononuclear cells (PBMC) of various sources, including from the patient to be treated. It is an important aspect of the present invention that the T cell to be activated derived from patient blood to be treated or from any other useful source. In the hospital or medical pathology laboratory, patient's blood can be obtained further can be isolated T cells and exposed T cells with the eDC epitope. In this invention eDC can be easily co-cultured with the patient's T cells to obtain viral protein-specific T cells further delivery back to the patient. However, it is known that some infected and sick patients have exhausted T cells, and some have exhausted T cells so severely that those person T cells not possible to activate. In this context, the present invention, for the first time, allows for activation of other sourced T cell populations to be activated by the eDCs of the invention, which can robustly activate T cells further can be administered to the patient in need of such treatment.

The eDCs and T cells are co-cultured in a T cell culture media, such as comprising RPMI 1640 Medium (Gibco, ThermoFisher Scientific) with 5% human serum (Sciencell Research Laboratories (Carlsbad, CA) or ImmunoCult™-XF T Cell Expansion Medium (StemCell Technologies). The thus obtained activated an proliferated T cells can then be administered to the patient in need.

Step 7: Therapeutic administration: The ex vivo/in vitro produced activated T cells are then administered to a patent to treat, stop or reduce a pathogenic infection, such as a viral infection.

The activated T cells can be administered to the patient in a pharmaceutically acceptable composition together with a pharmaceutically acceptable stabilizer, excipient and/or diluent, such as human albumin.

In one embodiment, the T cells are administered once to the patient, but in other embodiments are administered in multiple doses about 1 day, 2 days, 3 days, 4 days, 5 days, 6days 7 days, 8 days, 9 days, 10 drays, 11 days, 12 days, 13 days, 14 days, 15 days, 16 17 ays f 18 days, 19 days 20 ays, 21 days, a month after the initial administration.

The inventors examined the percentage of pure T cells with anti-CD4⁺ and -CD8⁺ T cell antibodies after the T cell expansion using flow cytometry. An initial quantity of 10×10⁶ T cells can usually generate 200×10⁶ T cells with 2-3 rounds of expansion. As established previously, a viral-specific T cell can be used for patients at 1×10⁶ cell/kg of body weight.²⁵ Inventor can optimize the method to ensure a robust expansion of T cells as patients need. Large-scale cGMP T cells can be biobanked for future use by the autologous patient or allogenic purposes. Ninety-four percent of mismatched patients receiving anti-viral T cells had no adverse or minimal graft versus host disease (GVHD).^{25,26} Thus, this inventive potent eDC-activated T cells compared to peptide-pulsed DCs can provide broader clinical applications for patients with no or exhausted T cells.

### EXAMPLES

### Example 1

### DC Differentiation Culture Protocol

The present inventors developed highly functional DCs from human induced pluripotent stem cells (HiPSCs) to fabricate reproducible and sensitive eDC probes. DCs are antigen-presenting cells capable of stimulating naïve T cells. Thus, DCs are pivotal in generating adaptive immunity and protecting against infections like SARS-CoV-2. We purchased commercially available genetically distinct HiPSC from three diverse sources to develop highly functional and reproducible DC. These HiPSCs originated from Creative Bioarray (Shirley, NY), MilliporeSigma (Burlington, MA), and Cell Application Inc. (San Diego, CA). We directly differentiated DC from HiPSC in four step-wise protocols:

STEP 1: Generation of a 3D spheroid with cGMP grade mTeSR plus medium StemCell Technologies) containing (cGMP grade media containing mesodermal growth factors and cytokines such as human recombinant (rh) bone morphogenetic protein 4 (BMP4), rh vascular endothelial growth factor (VEGF), rh stem cell factor (SCF), and rh granulocyte-macrophage colony-stimulating factor (GM-CSF) with 50 ng/mL concentrations for two days. We purchased all the growth factors from StemCell Technologies (Vancouver, Canada).

STEP 2: Generation of myeloid using cGMP grade StemSpan-AOF (StemCell Technologies) containing 50 ng/mL of rh BMP4, rh VEGF, rh SCF, and rh GM-CSF for two weeks. Typically, after the two days of culturing in Step 1, 50% of the medium is replaced with new medium, such as StemSpan-AOF which also contains rh BMP4, rh VEGF, rh SCF, and rh GM-CSF.

STEP 3: Immature DCs (iDC)s from myeloid cells by withdrawing BMP4, VEGF, and SCF time-dependently and adding IL-4 (100 ng/mL, StemCell Technologies) for one week in the culture. We ensure iDC expresses the CD209 marker over 90% in this step. Further, we treated iDC with maturation factor as step 4.

STEP 4: To the generation of mature DCs (mDC)s from iDC using cGMP grade StemSpan-AOF containing rhGM-CSF (50 ng/mL), rhIL-4 (100 ng/mL), rhTNF-α (50 ng/mL), rhIFN-γ (20 ng/ml), prostaglandin E2 (PGE2, 1 μg/ml), and rhIL-1β (10 ng/ml) for two days. FIG. 2A illustrates the morphology of HiPSC's undifferentiated colonies, 3D spheroids, and translucent myeloid to release iDC with protrusion. The induction of the maturation factor in iDC led to the outcome of a long veil and dendrites around the DC to obtain a fully functional mDC, which was ready to use for generating an eDC. All the maturation DC is verified with DC maturation marker protein via ICC and flow cytometry.

### Example 2

### DC Characterization Protocol

DCs are the immune cells capable of phagocytosing apoptotic cells, with cross-present viral, tumor, and self-antigens to T cells but not macrophages. In this context, we exposed our iDC to fluorescein isothiocyanate (FITC) IgG beads (Cayman Chemical, Ann Arbor, MI) for 24 hours. FITC IgG bead procedure was followed as per manufacturer protocols. We found iDC phagocytose beads>98%, but untreated DC showed no engulfed beads. FITC bead activity was determined by flow cytometry, contrast imaging, and fluorescence microscope (FIG. 2B). We also characterized our iDCs and mDCs with established DC markers to validate their functional capability to generate highly efficient eDC epitopes to activate T cells more efficiently. We used previously published DC markers: CD209⁺ (DC-Sign), CD40⁺, CD1c⁺, CD11C⁺, CD86⁺, MHC Class-II⁺ (MHC-II⁺), and HLA-DRA⁺ markers at >85% efficiency, which determined by immunocytochemistry (ICC) and flow cytometry as a previously established method.^{3,14,15} DC-Sign highly expressed iDC (99.5%) but decreased in mDCs (˜80%), as shown in our flow cytometry histogram (FIG. 3). Our mDCs expressed higher CD40⁺, CD11C⁺, and CD86⁺, as shown in ICC (FIG. 3A). In addition, the expression of MHC-II⁺ and HLA-DRA⁺ is vital for the antigen representation in mDCs that tightly regulates T cell activation via TCR. We found both MHC-II⁺ and HLA-DRA⁺ expressed in our mDCs>90%, which validates that our DCs are fully functional (FIG. 3B) and will have the ability to activate T cells efficiently and can have a novel immunotherapy platform by using these DCs.

### Example 3

### Fluorescent Imaging and Analysis

ICC and flow cytometry is a common cellular protein or antigen-detecting and visualization techniques that can recognize the target of interest or specific marker expression in cells via imaging and a series of flow histograms. The antibody is directly or indirectly linked to a reporter, such as a fluorophore or an enzyme. We performed ICC and flow cytometry as previously established method.^{14,15} Briefly, for flow cytometry, cells were collected further blocked with 2% mouse serum and staining with anti-CD209, CD40⁺, CD11C⁺, CD86⁺, MHC Class-II⁺ (MHC-II⁺) and HLA-DRA⁺ antibody (BD Biosceinces) as previously established method²⁷ and run to NovoCyte Flow Cytometer (Agilent Technologies, Santa Clara, CA). All the dilution of antibodies followed the manufacturer's guidelines. We have grown DC in a matrigel (Corning Inc) matrix to perform ICC on the 24-well plates. The further DCs were kept for one week to settle in gel matrix domes. After one week DC in domes was washed with PBS (phosphate-buffered saline) and fixed with 4% paraformaldehyde (PFA, Thermo Scientific). After additional washes in PBS, the DCs were permeabilized in 0.1% Triton X-100 (Sigma-Aldrich), followed by blocking with 5% BSA (bovine serum albumin) in PBS containing 10% normal goat serum (NGS, Thermofisher Scientific). Next, the DCs were incubated overnight with primary antibodies ((anti-CD209⁺ (DC-Sign), CD40⁺, CD1c⁺, CD11C⁺, CD86⁺, MHC Class-II⁺ (MHC-II⁺) and HLA-DRA⁺ antibodies)) diluted in a blocking solution at 4° C. with an appropriate dilution indicated by the manufacturer. The next day, the DCs were washed three times with washing buffer (1×PBS containing 0.05% Tween 20 and 1% NGS). Then DCs were incubated for 2 h at 25 ° C. with a fluorescent secondary antibody (Life Technologies). Further DCs were incubated with DAPI dye (Thermo Scientific) for 5 min, followed by the PBS wash, and visualized with a fluorescent microscope (ECHO Revolve Microscope, San Diego, CA). All antibodies were purchased from BD Biosciences or Invitrogen. NGL lab differentiated DCs from HiPSCs, which have all the marker proteins usually observed in human in vivo DCs.

### Example 4

### Genetically Engineered DC Generation Protocol

Inventor fully functional mDC was further aimed to be genetically engineered with various SARS-CoV-2 spike protein (Sp) fragments linked to the HLA-DRA molecule. We designed eight Sp constructs, where two were labeled with GFP to verify Sp expression via a fluorescence microscope, as shown in FIG. 4A-B. To do this, we first built a construct with the HLA-DRA molecule using lentivirus packaging to express the Sp with a fee-based service from VectorBuilder (Chicago, IL) to engineer the DC with various SARS-CoV-2-based Sp constructs, as shown in FIG. 4A. We received eight Sp constructs in lentiviral particles ready to use in our DC that expressed different Sp fragments.

To engineer our DC with various Sp constructs, we used a lentiviral vector of Sp with GFP epitopes (FIG. 4A) to DC at lentivirus concentrations of 1x10 6 transduction unit (TU) to DC count 1×10⁵ (1:10) using centrifugation at 1000×g for 2 h to obtain the desired transduction efficiency. We used Sp constructs with GFP to determine our Sp construct transduction efficiency in DC by fluorescence microscope and flow cytometry using a GFP channel. Once we determined Sp transduction efficiency over 80%, then our optimized DC engineering method was used in the overall experiment using various Sp constructs in DC.

Sp constructs used herein contain S1 and S2 regions of Sp, including the receptor binding domain (RBD) and receptor binding motif (RBM) to facilitate efficient integration into the DC genome resulting in significantly enhanced T cell activations. We successfully integrated each Sp fragment into the DC genome with ≥80% efficiency, as determined by flow cytometry, fluorescence microscope, and quantitative polymerase chain reaction (qPCR) (FIG. 4C-E). After genetically engineered DC (eDC) development using the Sp constructs, we cultured them with CD4⁺ T cells and compared eDC epitopes with Sp-pulsed DC. T cell proliferation increased 2-fold using our eDC epitope compared to Sp-pulsed DC (FIG. 4F). Thus, our eDC probe is highly sensitive and has higher specificity to expand T cells as the need for the reduction of SARS-CoV-2 and Long COVID significantly.

### Example 5

### CD4⁺ and CD8⁺ T cells isolation and proliferation ex vivo

Inventor initial development of six Sp constructs, excluding two GFP total of eight eDC epitopes, possesses the highest capability for activating CD4⁺ and CD8⁺ T cells with greater functionality than peptide-pulsed DCs. In this context, we exposed clinical samples with our six eDC epitopes. We purchased clinical PBMC samples from StemCell Technologies (Cambridge, MA) and RayBiotech Life, Inc. (Peachtree Corners, GA) covering healthy control (HC, n=9), COVID-19 (COV19, n=9), COVID-19 vaccinated healthy control (VHC, n=5) and Long COVID (LongCOV, n=5) clinical presentations (see results in Table 1). First, we isolated CD4⁺ and CD8⁺ T cells from PBMC using the EasySepTM human CD4⁺ and CD8⁺ isolation kit (StemCell Technologies) using the manufacturer's directions. We isolated CD4⁺ and CD8⁺ T cells with >90% efficiency (FIG. 5). CD4⁺ and CD8⁺ T cells were labeled with carboxyfluorescein diacetate succinimidyl ester (CFSE), and Vybrant DiD labeling kits (Invitrogen), respectively. The isolated CD4⁺ and CD8⁺ T cell numbers were determined by flow cytometry's FITC and APC-Cy7-A channels (FIG. 5A). IL-2 (100 pg/mL) was used in T cells in DC culture for establishing the positive selection of proliferative CD4⁺ and CD8⁺ T cells (Division 1-7), but negative controls were T cells without DC (Division 0), as shown in FIG. 5B. We used each assay's positive and negative control to calculate the rate of T cell frequencies from each eDC epitope of each clinical sample. We have used technical replicates of each clinical sample for all experiments to provide results with higher accuracy.

TABLE 1
Clinical sample for testing engineered DC

| | | COVID-19 | | |
|---|---|---|---|---|
| | | Vaccinated | | Chronic |
| | HEALTHY | healthy | | COVID-19 |
| | CONTROL | control | COVID-19 | (Long |
| CONDITION | (HC) | (VHC) | (COV19) | COVID) |
| CD+ | n = 9 | n = 5 | n = 9 | n = 5 |
| T cells | | | | |
| CD8+ | n = 5 | n = 5 | n = 5 | n = 5 |
| T cells | | | | |

### Example 6

### CD4+T Cell Activation With eDC Epitopes

We introduced our six basic eDC Sp epitopes without GFP, including spike (Spep), membrane (Mpep), and nucleocapsid (Npep) peptide-pulsed DC to our CD4⁺ and CD8⁺ T cell cultures and incubated them for seven days to determine the frequency of T cells (Division 1-7). We observed that VHC, COV19, and LongCOV subjects had higher responses with a higher frequency of CD4⁺ and CD8⁺ T cells with our eDC epitopes compared to various SARS-CoV-2 peptide-pulsed DCs such as Spep, Mpep, and Npep (FIG. 6A). Our eDC epitopes developed from Sp of S1 and S2 regions of SARS-CoV-2, similar to the Spep pulsed fragments. Thus, we compared all our eDC epitopes with Spep and found that eDC-2, eDC-5, and eDC-7 had a significantly higher response on T cell frequencies than Spep. More interestingly, the Si subunit of HLA-DRA molecule combination eDC-2 showed significantly higher responses (p<0.05) on activating CD4⁺ T cells in all clinical samples (FIG. 6B) using One-way ANOVA Dunn's multiple comparisons tests (GraphPad Prism, San Diego, CA). The analysis of CD4⁺ T cell activation data of eDC-2 epitopes indicates that it is the likely epitope for treating SARS-CoV-2 compared to the traditionally DC pulsed peptide. In addition, we also confirmed that the HLA-DRA molecule attached in the eDC-2 construct led to higher T cell frequency than the eDC-3 epitopes without HLA-DRA, as demonstrated the Sp construct linked with HLA-DRA molecule is a novel eDC product for improving immunity (FIG. 4F). DC naturally represents HLA-DRA⁴ but constructs with an additional molecule with HLA-DRA-boosted T cell responses, as observed in our clinical samples, which is unique to our eDCs. Thus, we can generate eDC epitopes with any viral protein construct and produce viral-specific protective T cells to treat current and newly existing viruses to prevent pandemics like COVID-19.

### Example 7

### CD8⁺ T Cell Activation With eDC Epitopes

We also explored the CD8⁺ T cells due to their sustained immunity in patients with a chronic viral infection. Stem-like CD8⁺ memory T cells exist in convalescent SARS-CoV-2 individuals, and their presence is possibly indicative of long-term protection against the virus, which can be beneficial like CD4⁺ T cells. 28 Thus, we cultured each clinical CD8⁺ T cell with our eDC epitopes and peptide-pulsed DC sample. We found CD8⁺ T cell frequencies with our epitopes substantially decreased in LongCOV subjects but increased in the VHC and COV19 samples compared to the HC, as shown in FIG. 7. Our data indicated that LongCOV exhausted CD8⁺ T cells, which might be caused due to their prolonged fight against infection usually observed in the chronic infection population.^{16,29} We also found CD8⁺ T cell response with eDC-2, eDC-5, and eDC-7 increased>2-4 folds depending on the epitopes compared to Spep (FIG. 7A). In addition, using One-way ANOVA Dunn's multiple comparisons tests, the eDC-2 epitope again showed a significantly higher response in CD8⁺ T cell activation compared to Spep in HC and COV19 samples. LongCOV still showed a higher T cell response than Spep (FIG. 7B), even though statistically insignificant may be due to the higher exhaustion of CD8⁺ T cells in chronic infection. Our overall data indicate that eDC-2 is a highly efficient product elevating CD4⁺ and CD8⁺ T cell frequencies in all clinical samples regardless of T cell exhaustion. Thus, generating probes like eDC2 with M, N, and ORFs of SARS-CoV-2 can be preventive for multi variants of SARS-CoV-2 and chronic infection. Thus, our novel eDC-2 product can target various SARS-CoV-2 proteins besides Sp and prevent the omicron or delta variants that no vaccine can prevent.

### Example 8

### Intracellular IFN-γ⁺ CD4⁺ Detection by Flow Cytometry

Activation of T cells results in intracellular expression and secretion of cytokines such as IFN-γ, established in various clinical studies.^{16,17,18} It is well known that the activated T cells produce intracellular IFN-γ significantly higher than non-activated samples. Therefore, we investigated whether our activated T cells by our eDC epitopes can secrete IFN-γ, which led to measuring intracellular IFN-γ⁺ using anti-IFN-γ antibodies (BD Biosciences) with flow cytometry. To determine intracellular IFN-y+, we have collected T cells (1×10⁶ T cells) from treated T cells with eDC or IL-2 or peptide-pulsed DCs. Further T cells were washed with PBS, followed by the T cells blocking with 2% NMS (normal mouse serum) in PBS (NMS, Thermofisher Scientific). Further T cells were permeabilized in 0.1% Triton X-100 (Sigma-Aldrich) and washed thrice with PBS. After that DCs were incubated 2 h with primary antibodies (anti-IFN-γ antibodies, BD Biosciences) diluted in a blocking solution at 4° C. with an appropriate dilution indicated by the manufacturer. DCs were washed three times with washing buffer (1×PBS containing 0.05% Tween 20 and 0.5% BSA). Then DCs were incubated for 1 h at 25° C. with a fluorescent secondary antibody (Life Technologies). Our eDC epitopes induced higher levels of IFN-γ expression, specifically with the eDC-2, eDC-5, eDC-7, and eDC-8 epitopes in HC, VHC, and COV19 samples, compared to the spike protein pulsed DC (Spep), as shown in FIG. 8A. All our eDC epitopes were compared to the Spep to perform a statistically significant comparison (*p<0.05 and **p<0.01). FIG. 8B represented the flow cytometer histograms of COV19 samples where eDC-2 had a higher response (arrow) than any other epitope of eDC, including Spep. We further confirmed our intracellular IFN-γ flow cytometry data with IFN-γ secretion in CD4⁺ T cell media via ELISA kit (StemCell Technologies). Thus, IFN-γ secretion in our T cells was significantly higher with eDC epitopes which validates that our activated T cells are highly functional and patients will benefit from our genetically engineered T cell probe-based therapy

### Example 9

### Cytokine Secretion from eDC-Activated CD4⁺ T Cells by ELISA

To determine functionally committed T cells, we have measured IFN-γ, IL-2, and TNF-α using ELISA kits from StemCell Technologies and BioLegends (San Diego, CA). We collected seven days of cultured T cell supernatant which was initially treated with eDC epitope and other peptides. Further, the supernatant was diluted, processed, and performed ELISA assays using manufacturer protocols. Before measuring the absorbance, we used a stop solution provided with the ELISA kit and then read the absorbance with a microplate reader (SpectraMax iD3 Reader, Molecular Devices, San Jose, CA). Our ELISA results showed all cytokines, such as IFN-γ, IL-2, and TNF-a increased in SARS-CoV-2 patients.^{16,17,18} We also found that IFN-γ, IL-2, and TNF-α were significantly elevated in the COV19 and LongCOV patient samples compared to HC and VHC (FIG. 9A). Interestingly, IL-2 substantially increased in all the clinical samples in higher levels (>200 pg/mL) compared to IFN-γ and TNF-α (<100 pg/mL) as shown in FIG. 9B. We also compared our eDC epitopes-based IFN-γ, IL-2, and TNF-α secretion with Spep and found that most of our eDC epitopes (eDC-2, eDC-5, eDC-7, and eDC-8) secrete significantly higher levels of cytokines compared to Spep. Moreover, Spep had no significant difference in secreting these cytokines compared to non-treated control samples. But our eDC epitopes secreted these cytokines significantly higher than control samples which validate that our innovative eDC epitopes are superior to any peptide-pulsed DC used in traditional T cell proliferation studies.^{16,17,18} Our eDC-2 results consistently showed significantly higher levels of IFN-γ secretion (p<0.05) in both cellular and media fractions in CD4⁺ T cells (FIGS. 8-9). Overall results indicated that eDC-2 is highly sensitive in secreting increased levels of IFN-γ (>25 pg/mL), IL-2 (>200 pg/mL), and TNF-α (>35 pg/mL) compared to any other eDC epitopes and Spep, which validates the use of eDC epitopes for efficient T cell therapy. TNF-α significantly showed an increase in COV19 and LongCOV samples by eDC-2, which important proinflammatory cytokines usually increased in infections (FIG. 9C). DC naturally represents HLA-DRA⁴, but our innovative approach to developing eDC constructs with an additional molecule with HLA-DRA-boosted T cell responses which observed in our clinical samples. Thus, using our eDC epitopes with M, N, and ORFs construct similar to the eDC-2 epitope to provide broader protection against the new variant of SARS-CoV-2, such as Omicron and Delta, or any other newly mutated viruses.

### Example 10

### In-Vitro MTT Cell Viability Assay

We determined DCs viability after the DC transduction (genetically modification by a viral vector) with Sp constructs to understand whether genetically engineered DC (eDC) reduced its viability. In this context, we collected eight eDCs mixed T cell cultures and plated them in a 24-well plate. MTT [3-(4,5-dimethylthiazol-2-yl)-2,5-diphenyltetrazolium bromide] assay is a colorimetric assay to evaluate cell metabolic activity following the manufacturer's instructions for the assay (MilliporeSigma). To perform the reported established MTT assay³⁰, we added 1 mg/mL of MTT reagent (MilliporeSigma) to each well eDCs mix T cell culture, followed by 2 h incubation at 37° C. Further, carefully remove the MTT from the mixed culture, adding dimethyl sulfoxide (DMSO) in each -well and mixed. After the 10 min incubation, DMSO absorbed the MTT, followed by the careful transfer of DMSO absorbed MTT without removing any cells in the fresh plate and run for a plate reader (SpectraMax iD3 Plate Reader, Molecular Devices, San Jose, CA) with the optical absorption density (OD) of 570 nm to determine the amount of MTT binding in the viable cell as shown in FIG. 10. This method determined our eDC mixed cells viability compared to DC mixed cells without genetic modification (untreated) by Sp constructs of SARS-CoV-2. We calculated the amount of MTT in each well using the following formula:

$$Cell Viability ( \% ) = \frac{OD 570 treated D C mix}{OD 570 control D C mix} \times 100$$

### Example 11

### Discussion

The present inventors successfully developed an innovative genetically engineered DC that activates significant levels of CD4⁺ and CD8⁺ T cells to generate protective T cell therapy against new and existing viral and other pathogenic infections, such as SARS-CoV-2 variants and chronic infections. To develop a highly efficient genetically engineered DC epitope (eDC), we first generated fully functional cGMP grade naive DCs from HiPSCs (FIG. 2A), which can be used for training Sp-specific T cells from the patient in vitro. Our DC are directly differentiated from HiPSCs on a robust and cGMP scale. We established that our DC is highly functional and can phagocytose the beads with a capability of over 90%, which validates our DC is fully functional (FIG. 2B). Immature and mature DC markers were established to determine DC's extensive functional activity and morphological characteristics with CD209⁺ (DC-Sign), CD40⁺, CD1c⁺, CD11C⁺, CD86⁺, MHC Class-II⁺ (MHC-II⁺), and HLA-DRA⁺ markers at >85% efficiency (FIG. 3A). Moreover, our flow cytometry results showed that HLA-DRA⁺ and MHCII⁺ expression was over 90% (FIG. 3B), crucial molecules for antigen-presenting cells like DCs to bind with T cell receptor (TCR) to activate CD4⁺ and CD8⁺ T cells. These highly functional DCs were then ready to genetically engineer with Sp constructs. We found our DCs transduced with various Sp epitopes with 80% efficiency (FIG. 4).

Once NGL established the eDC, its capability to activate T cells was further examined and verified. We purchased clinical PBMC to isolate CD4⁺ and CD8⁺ T cells. We successfully demonstrated CD4⁺ and CD8⁺ T cell isolation processes from clinical PBMC with >90% efficiency (FIG. 5). NGL's eDC with Sp linked with HLA-DRA epitopes are highly functional and activate CD4⁺ and CD8⁺ T cells of COVID-19 patients significantly compared to traditional peptide-pulsed DC in all clinical samples (FIGS. 6-7). Our genetically engineered SARS-CoV-2 protein-specific eDC-activated CD4⁺ and CD8⁺ T cells also can spontaneously secrete IFN-γ, IL-2, and TNF-α which validate functionally committed T cells production (FIGS. 8-9). We found eDC treated CD4⁺ T cells secretes significantly (p<0.05) higher levels of IFN-γ, IL-2, and TNF-α compared to untreated cells (without eDC) in COVID-19 and Long COVID clinical samples using One-way ANOVA Dunn's multiple comparisons tests (FIG. 9). We found eDC-2 epitopes significantly increased T cell frequencies similar to elevated levels of IFN-γ, IL-2, and TNF-α secretion specifically in SARS-CoV-2 patients. Our overall eDC epitopes showed functionally committed T cells with higher cytokine secretion over the peptide-pulsed DC. Moreover, the eDC-2 epitope possessed a high potential for enhancing functional T cells compared to other eDC epitopes. Moreover, we have tested eDC and T cell mix culture cell viability using MTT assay. Our results showed all cell viability over 80%, which demonstrated our eDC and its process in activating T cells are completely safe (FIG. 10). Thus, our eDC-2 epitope provides a therapeutic potential for COVID-19 patients with exhausted T cells and improves their immunity.

With our eDC epitope's robust response to activating functional T cells, we understand eDCs are not limited to Sp-specific as Pfizer or Moderna vaccines but can be used for M and N or other viral protein-specific immunity. So, we also exposed CMV and BK viral proteins to our DC. We found a robust and significant response (p<0.05) in the Long COVID patient's CD4⁺ T cells with increased levels of IFN-y secretion that can be used for treating Long COVID and transplantation-related viral infections. Thus, our genetic engineer DC with various SARS-CoV-2 epitopes ((M, N, and open reading frames (ORFs)), including transplantation of infection-related viral protein, can be used in diagnosis and therapy against a broad spectrum of SARS-CoV-2, CMV, and BK viruses to prevent current COVID-19 or transplantation-related viral infections.

### Characteristics and Improvement of the Present Invention

Direct differentiation of DC from HiPSC in cGMP grade provides clinical potential compared to the traditional DC generation method, which requires a feeder layer of hematopoietic stem cells that usually come from animal cells.

Robust DC (iDC and mDC) marker expression over 85% validates its use for engineering DC further use for T cell activation efficiently. All the DC protein expressions, like in vivo human DC, validate its capability to activate protective T cells against viral or degenerative disorders.

Genetically engineered DC (eDC) transduction efficiency with spike protein of SARS-CoV-2 was over 80% without reducing cell viability, validating our innovative eDC's safe clinical uses.

Inventor demonstrated the eDC-linked HLA-DRA molecule-based epitope's high functionality (p<0.05) by significantly activating and expanding clinical CD4⁺ and CD8⁺ T cells compared to usual peptide-pulsed DCs. Usually, peptide-pulsed DCs are used for T cell activation for immune boosting. But eDC epitopes linked with HLA-DRA molecule provide a significantly higher immune response than the standard traditional method that validates eDC superiority for efficient T cell therapy purposes.

Sp transduced eDC significantly elevated the frequency of SARS-CoV-2 specific protective CD4⁺ and CD8⁺ I cells in COV19 and LongCOV patients; those have exhausted T cells reported in various publications.^{5,6} Thus, eDC epitopes provide the chance to improve protective T cells if patients exhausted T cells severely, which no other traditional method or vaccine can do.

The innovative eDC co-cultured with CD4⁺ and CD8⁺ T cells, which occurred in ex vivo, thus avoiding in vivo immune stimulation in risk groups. Therefore, in the hospital or medical pathology laboratory, our eDC epitope will be easy to co-culture with patient's T cells and obtain viral protein-specific T cells for delivery back to the patient. Thus, the production of viral protein-specific protective CD4⁺ and CD8⁺ T cells will almost occur at natural levels. This will not cause over immune stimulation in vivo, thus providing an efficient T cell immune-boosting platform.

The novel eDC is not limited to SARS-CoV-2 protein. Suppose patients lack specific viral-based protective T cells. In that case, we can engineer our DC with this specific pathogenic or degenerative protein to expand T cell ex vivo and further deliver to patients to improve their T cell immunity. To expand our innovative eDC approach, we also exposed CMV, and BKV proteins to our DC culture, which significantly elevated functionally committed IFN-γ⁺CD4⁺ T cells in the chronic SARS-CoV-2 (LongCOV) patient samples (FIG. 11A-B). These results indicate the future use of eDC epitopes using CMV, BKV, and other potential viral protein-specific constructs to treat transplantation-related infections and LongCOV.

The innovative eDC-activated T cell therapy approach is safe for patients due to their own blood-based product providing easy access for clinical use. eDCs can easily be removed from T cell culture via filtration. Thus, only autologous T cells will be delivered to the patient, which is extremely safe clinical use for T cell therapy for current and newly existing pathogenic bacteria, viruses, and other degenerative proteins like toxic amyloid-β (Aβ) or tau proteins that cause Alzheimer's disease as we observed previously.

No autologous eDC ex vivo T cell stimulation currently exists. Therefore, the present invention provides a multiepitope-based eDC probe (T cell activator) with various viral proteins that can be widely used to diagnose and treat transplants, immunosuppressed, chronic illnesses, cancer patients with compromised immune systems, and older patients.

The eDCs of the invention when exposed to T cells provide a robust activation response, as compared to prior peptide pulsed DCs, thereby ensuring that patients who lace T cells or sufficient T cell response, can be successfully treated by ex vivo generation of activated T cells that can then be administered to the patients in need of treatment.

The eDCs of the invention are prepared from naïve DC cells from HiPSCs, which are highly functional as compare to those from some sick or impaired patients (such as elderly patients)^{31,32,33}, thus providing an improved therapeutic potential as compared to DCs obtained from the blood or bone marrow from such impaired patients.

### REFERENCE CITED IN THE SPECIFICATION

ADDIN Mendeley Bibliography CSL_BIBLIOGRAPHY 1. COVID-19 death in US. Accessed May 2022. https://covid.cdc.gov/covid-data-tracker/#datatracker-home.

2. Current COVID-19 status. Accessed May 6, 2023. https://covid.cdc.gov/covid-data-tracker/#datatracker-home.

3. Begum, A. N. et al. Women with the Alzheimer's risk marker ApoE4 lose Aβ-specific CD4+ T cells 10-20 years before men. Transl. Psychiatry 4, (2014).

4. McKinney, D. M. et al. A strategy to determine HLA class II restriction broadly covering the DR, DP, and DQ allelic variants most commonly expressed in the general population. Immunogenetics 65, 357-370 (2013).

5. Roe, K. A role for T-cell exhaustion in Long COVID-19 and severe outcomes for several categories of COVID-19 patients. J. Neurosci. Res. 99, 2367-2376 (2021).

6. Rha, M. S. & Shin, E. C. Activation or exhaustion of CD8+ T cells in patients with COVID-19. Cell. Mol. Immunol. 18, 2325-2333 (2021).

7. Xu, Z. et al. Xu Z, Shi L, Wang Y, et al. Pathological findings of COVID-19 associated with acute respiratory distress syndrome [published correction appears in Lancet Respir Med. 2020 Feb. 25;]. Lancet Respir Med. 2020; 8 (4):420-422. doi:10.1016/52213-2600(20)30076-X. Lancet Respir Med. 8, 420-22 (2020).

8. Calvet, J. et al. Cd4 and cd8 lymphocyte counts as surrogate early markers for progression in sars-cov-2 pneumonia: A prospective study. Viruses 12, (2020).

9. Riou, C. et al. Profile of SARS-CoV-2-specific CD4 T cell response: relationship with disease severity and impact of HIV-1 and active Mycobacterium tuberculosis co-infection. medRxiv 2021.02.16.21251838 (2021).

Trougakos, I. P. et al. Adverse effects of COVID-19 mRNA vaccines: the spike hypothesis. Trends Mol. Med. 28, 542-554 (2022).

11. Anand, P. & Stahel, V. P. Review the safety of Covid-19 mRNA vaccines: a review. Patient Saf. Surg. 15, 1-9 (2021).

12. Howard, C. J., Charleston, B., Stephens, S. A., Sopp, P. & Hope, J. C. The role of dendritic cells in shaping the immune response. Anim. Heal. Res. Rev. 5, 191-195 (2004).

13. What are the dendritic cells. Accessed May 24, 2023. https://www.news-medical.net/health/What-are-Dendritic-Cells.aspx.

14. Begum, A. N. et al. Rapid generation of sub-type, region-specific neurons and neural networks from human pluripotent stem cell-derived neurospheres. Stem Cell Res. (2015) doi:10.1016/j.scr.2015.10.014.

Begum, A. N., Aguilar, J. S., Elias, L. & Hong, Y. Silver nanoparticles exhibit coating and dose-dependent neurotoxicity in glutamatergic neurons derived from human embryonic stem cells. Neurotoxicology (2016) doi:10.1016/j.neuro.2016.08.015.

16. Jin, H. T. et al. Cooperation of Tim-3 and PD-1 in CD8 T-cell exhaustion during chronic viral infection. Proc. Natl. Acad. Sci. U.S.A. 107, 14733-14738 (2010).

17. Littlefield, K. M. et al. SARS-CoV-2-specific T cells associate with inflammation and reduced lung function in pulmonary post-acute sequalae of SARS-CoV-2. PLoS Pathog. 18, 1-20 (2022).

18. Karki, R. et al. Synergism of TNF-α and IFN-γ Triggers Inflammatory Cell Death, Tissue Damage, and Mortality in SARS-CoV-2 Infection and Cytokine Shock Syndromes. Cell 184, 149-168.e17 (2021).

19. Scherlieβ, R. The MTT assay as tool to evaluate and compare excipient toxicity in vitro on respiratory epithelial cells. Int. J. Pharm. 411, 98-105 (2011).

20. Kumar, P., Nagarajan, A. & Uchil, P. D. Analysis of cell viability by the MTT assay. Cold Spring Harb. Protoc. 2018, 469-471 (2018).

21. Ku, M. W. et al. Lentiviral vector induces high-quality memory T cells via dendritic cells transduction. Commun. Biol. 4, 1-16 (2021).

22. Tang, L. et al. Enhancing T cell therapy through TCR-signaling-responsive nanoparticle drug delivery. Nat. Biotechnol. 36, 707-716 (2018).

23. Garnier, L. et al. IFN-γ-dependent tumor-antigen cross-presentation by lymphatic endothelial cells promotes their killing by T cells and inhibits metastasis. Sci. Adv. 8, 1-19 (2022).

24. Wu, B. et al. RNA polymerase II pausing factor NELF in CD8+ T cells promotes antitumor immunity. Nat. Commun. 13, 1-14 (2022).

25. Hague, T. et al. Treatment of Epstein-Barr-virus-positive post-transplantation lymphoproliferative disease with partly HLA-matched allogeneic cytotoxic T cells. Lancet 360, 436-442 (2002).

26. Melenhorst, J. J. et al. Allogeneic virus-specific T cells with HLA alloreactivity do not produce GVHD in human subjects. Blood 116, 4700-4702 (2010).

27. Spence, J. R. et al. Directed differentiation of human pluripotent stem cells into intestinal tissue in vitro. Nature 470, 105-110 (2011).

28. Kared, H. et al. SARS-CoV-2-specific CD8+ T cell responses in convalescent COVID-19 individuals. J. Clin. Invest. 131, 1-13 (2021).

29. Hashimoto, M. et al. CD8 T Cell Exhaustion in Chronic Infection and Cancer: Opportunities for Interventions. Annu. Rev. Med. 69, 301-318 (2018).

30. Grabinger, T. et al. Ex vivo culture of intestinal crypt organoids as a model system for assessing cell death induction in intestinal epithelial cells and enteropathy. Cell Death Dis. e1228-10 (2014).

31. Grolleau-Julius, A., Harning, E. K., Abernathy, L. M. & Yung, R. L. Impaired dendritic cell function in aging leads to defective antitumor immunity. Cancer Res. 68, 6341-6349 (2008).

32. Agrawal, A. & Gupta, S. Impact of aging on dendritic cell functions in humans. Ageing Res. Rev. 10, 336-345 (2011).

33. Clements, S. J., Maijo, M., Ivory, K., Nicoletti, C. & Carding, S. R. Age-Associated Decline in Dendritic Cell Function and the Impact of Mediterranean Diet Intervention in Elderly Subjects. Front. Nutr. 4, (2017).

1. A genetically engineered dendritic cell comprising a vector comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

2. The genetically engineered dendritic cell according to claim 1, wherein said pathogenic protein is a viral surface or membrane protein.

3. The genetically engineered dendritic cell according to claim 1, wherein said protein is a viral protein from SARS-CoV 2, cytomegalovirus (CMV), BK virus (BKV).

4. The genetically engineered dendritic cell according to claim 3, wherein said protein is the SARS-CoV 2 spike protein, or a fragment thereof.

5. The genetically engineered dendritic cell according to claim 4, wherein protein is at least one of the S1 and S2 subunit of SARS-CoV2, or a fragment thereof.

6. A kit comprising genetically engineered dendritic cells according to claim 1.

7. A kit comprising genetically engineered dendritic cells according to claim 1, wherein said dendritic cells are human dendritic cells derived from HiPSCs.

8. A polynucleotide construct comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

9. The polynucleotide construct according to claim 8, wherein said pathogenic protein is a viral surface or membrane protein. (Original) The polynucleotide construct according to claim 8, wherein said protein is a viral protein from SARS-CoV 2, cytomegalovirus (CMV), BK virus (BKV).

11. The polynucleotide construct according to claim wherein said vial protein is the spike peptide (Spep) from SARS-CoV 2, or a fragment thereof.

12. The polynucleotide construct according to claim 11, wherein the viral protein is at least one of the S1 and S2 subunits of SARS-CoV2 or a fragment thereof.

13. A method for producing genetically engineered dendritic cells, the method comprising:
(a) culturing human induced pluripotent stem cells (HiPSCs) in a first culture medium comprising mesodermal growth factors, consisting of human recombinant (rh) bone morphogenetic protein 4 (BMP4), rh vascular endothelial growth factor (VEGF), rh stem cell factor (SCF), and rh granulocyte-macrophage colony-stimulating factor (GM-CSF) to produce 3-dimensional spheroid cells;
(b) adding further factors to said first culture medium, wherein said further factors comprise rh BMP4, rh VEGF, rh SCF, and rh GM-CSF;
(c) separate the 3-dimensional spheroid cells from said further factors, and culture said 3-dimensional spheroid cells in a second culture medium that comprises IL-4 to produce immature dendritic cells (iDCs);
(d) separate said iDCs from said second culture medium and culture said iDCs in a third culture medium comprising rhGM-CSF, rhlL-4, rhTNF-α, rhIFN-γ, prostaglandin E2 (PGE2), and rhIL-1β to produce 3 dimensional spheroids of fully functional mature dendritic cells (mDCs); and
(e) transfecting said mDCs dendritic cell with a vector comprising (a) a polynucleotide sequence encoding a pathogenic or degenerative protein or fragment thereof and (b) a polynucleotide sequence encoding human leukocyte antigen—DR or -DRA (HLA-DR or HLA-DRA) or a fragment thereof.

14. The method according to claim 13, wherein said pathogenic protein is a viral surface or membrane protein.

15. The method according to claim 13, wherein said protein is a viral protein from SARS-CoV 2, cytomegalovirus (CMV), BK virus (BKV).

16. The method according to claim 15, wherein said protein is the spike peptide (Spep) from SARS-CoV 2, or a fragment thereof.

17. The method according to claim 16, wherein protein is at least one of the S1 and S2 subunit of SARS-CoV 2, or a fragment thereof.

18. A method for producing protein-specific T cells which comprises co-culturing T cells with genetically engineered dendritic cells according to claim 1.

19. The method according to claim 18, wherein said co-cultured T cells are obtained from a patient.

20. The method according to claim 18, wherein said co-cultured T cells are from a source other than a patient to be treated with said protein-specific T cells.

21. A method for treating a patient having a pathogen infection that comprises administering to a patient an effective amount of protein-specific T cells produced according to claim 18.

22. The method according to claim 21, wherein said protein-specific T cells are administered intravenously to said patient.

23. A T cell treatment preparation comprising protein-specific T cells according to claim 18 and a pharmaceutically acceptable carrier, diluent or medium.
